(19) United States
(12) Patent Application Publication
(10) Pub. No.: US 2015/0297060 A1
(43) Pub. Date: Oct. 22, 2015

(54) ENDOSCOPE PROPULSION

(71) Applicant: Edward Via College of Osteopathic Medicine, Blacksburg, VA (US)

(72) Inventor: M. Jonathan BERN, Roanoke, VA (US)

(73) Assignee: Edward Via College of Osteopathic Medicine, Blacksburg, VA (US)

(21) Appl. No.: 14/693,537

(22) Filed: Apr. 22, 2015

Related U.S. Application Data
(60) Provisional application No. 61/982,658, filed on Apr. 22, 2014.

(51) Int. Cl. A61B 1/00; A61B 1/31

(52) U.S. Cl. CPC A61B 1/0016; A61B 1/00154; A61B 1/31

(57) ABSTRACT

The present invention provides a system and method for active propulsion of devices, such as endoscopes, along cavities, such as body lumens. The propulsion system can be attached to a commercially available endoscope, or be provide affixed together, and moves the endoscope in a lumen by pulling it forward. The present invention further provides a method of diagnosing diseases and disorders, and treatment of diseases and disorders, using a device according to the invention.

### CROSS-REFERENCE TO RELATED APPLICATIONS

This application claims priority to U.S. Provisional Application No. 61/982,658, filed Apr. 22, 2014, whose disclosure is hereby incorporated by reference in its entirety into the present disclosure. The subject matter of the present application is related to that of U.S. Pat. No. 7,708,687, issued May 4, 2010, whose disclosure is hereby incorporated by reference in its entirety into the present disclosure. Any component of the system of the above-identified disclosures, and any step of the method thereof, may be incorporated into the present invention, including without limitation the details of the motor, drive cable, and drive gear.

### BACKGROUND OF THE INVENTION

1. Field of the Invention

The present invention relates to the field of health care. More specifically, the invention relates to the field of endoscopy, and particularly to devices and methods for performing endoscopic examinations and surgeries.

2. Description of Related Art

Each year, 60,000 Americans die from colon cancer, making colon cancer the second leading cause of cancer death in the United States. Early detection of the disease greatly improves survival. Furthermore, removal of pre-cancerous polyps can be achieved endoscopically, which prevents colon cancer altogether. Unfortunately early colon cancer and polyps are asymptotic. For this reason screening tests are needed to detect and prevent colon cancer. Currently available screening tests include fecal occult blood test, flexible sigmoidoscopy, and colonoscopy. In part because of the limitations of these tests, only about 10% of the United States population is currently screened for this common preventable cause of death.

Fecal occult blood testing detects blood in the stool that can not be seen on visual inspection of the stool. Unfortunately only about 30% of colon cancers can be detected by fecal occult blood testing, making this test too insensitive for effective screening.

Flexible sigmoidoscopy is a type of endoscopy that uses a semi-rigid tube with fiberoptic lenses to directly visualize the colon. The end of this semi-rigid tube has a flexible steering section to direct the instrument's tip. In an ideal patient, this test can visualize up to 60 centimeters of the distal colon (or approximately one-third of the entire colon). The limited extent of the flexible sigmoidoscopy exam misses approximately 50% of colon cancers. Although flexible sigmoidoscopy is insensitive, it is relatively inexpensive and can be performed as a screening test in a physician's office. Unfortunately flexible sigmoidoscopy is too uncomfortable for many patients to tolerate. Flexible sigmoidoscopy is painful because the scope is advanced in the colon by pushing the semi-rigid tube against the colon wall. As the tube is pushed against the colon wall, the colon is stretched. Stretching of the colon causes intense visceral pain. In addition to pain, stretching the colon too far can result in colon perforation, a potentially life threatening complication of flexible sigmoidoscopy.

Colonoscopy, like flexible sigmoidoscopy, is a type of endoscopy that utilizes a semi-rigid tube with either fiberoptic lenses or a video camera to directly visualize the colon. Currently available colonoscopes offer an excellent view of the colon. In a fashion similar to flexible sigmoidoscopy, the semi-rigid tube has a flexible steering section at the distal end of the instrument. Unlike the flexible sigmoidoscope, the colonoscope is long enough to visualize the entire colon. For this reason colonoscopy is ideal for colon cancer screening. If a pre-cancerous colon polyp is detected at the time of colonoscopy it can be removed through the scope's “working channel” using various endosurgical instruments (such as biopsy forceps and polypectomy snares). In a fashion similar to flexible sigmoidoscopy, pushing the semi-rigid tube against the colon wall advances the colonoscope. Unfortunately colonoscopy is far too uncomfortable to be performed without high level intravenous sedation or general anesthesia. The pain experienced during colonoscopy is related to stretching of the colon wall as the colonoscope is advanced. Colon perforation can occur as a result of pushing the semi-rigid tube too forcefully against the colon wall as the colonoscope is advanced. The high level of sedation needed for colonoscopy requires a highly monitored environment, such as an operating room. With the added operating room charges colonoscopy becomes quite costly. If colonoscopy were less expensive, it would be more widely accepted as a colon cancer-screening test.

Various robotic endoscopy devices and methods have been previously disclosed. Several such disclosures involve robotic endoscopes that are generally complex devices with multiple interacting segments. These robotic endoscopes generally involve a kinematically redundant robot, which generally has about seven or more internal degrees of freedom. These robotic endoscopes are also designed to function autonomously as a robot. That is, an examining physician has no direct control of the robotic endoscope. Furthermore, the examining physician can not directly assist in the movement of the scope in an organ lumen. The lack of direct physician control will markedly increase the risks of robotic endoscopy.

The previously disclosed robotic endoscopes also depend on a complicated interaction of a plurality of segments. At least one previous disclosure involves a robotic endoscope that relies on a complex array of pressure sensors, gripping devices, and expansion modules under the control of at least one computer. Even the slightest malfunction of the complex control mechanism could cause devastating complications for a patient.

More specifically, the prior robotic endoscope uses a proximal and a distal toroidal balloon in conjunction with an extensor module. The proximal toroidal balloon expands to statically grip the organ wall and thereby fix this segment of the robotic endoscope to the organ wall. After the proximal balloon has expanded, the extensor module expands, thus lengthening the robotic endoscope. The robotic endoscope depends primarily on the extensor module for movement. After the extensor module has lengthened the robotic endoscope, the distal toroidal balloon expands to fix this segment of the robotic endoscope to the organ lumen wall. After distal toroidal balloon inflation, the proximal toroidal balloon deflates and the extensor module contracts. This arrangement is said to produce an inch-worm-like movement in an organ lumen.

The toroidal balloon described in at least two such prior disclosures operates by means of static friction. This static friction is fundamental to the operation of the robotic endoscope. This static friction is between the balloon and organ wall. The only dynamic feature of the toroidal balloon's operation is expansion and contraction. Extension and contraction of the extensor module causes movement of the robotic endoscope in an organ lumen. As such, the extensor module is the main dynamic component of the robotic endoscope.

The toroidal balloon(s) described in at least these two prior disclosures involve a relatively small surface area. Thus high inflation pressures may be required to grip and fix the toroidal balloon to the organ wall. A high inflation pressure used to fix the toroidal balloon to an organ wall may distend the organ wall. Depending on the degree of organ wall distention, the patient may experience intense visceral pain. Therefore, robotic endoscopy according to these prior devices and methods may often require high level sedation or general anesthesia to permit a comfortable examination. In this regard, robotic endoscopy according to these prior disclosures offers no additional benefits to currently available endoscopic procedures.

Furthermore, the extensor module of these prior robotic endoscope disclosures is constantly changing the axial length of the robotic endoscope. As the robotic endoscope is constantly changing length, currently available endosurgical devices, such as biopsy forceps or polypectomy snares, may be very difficult if not prevented from conjunctive use.

The mechanical complexity of this prior approach and the need for computer control systems generally relate to relatively high production cost for the robotic endoscope. And, as in many fields, high production cost could substantially limit the availability of robotic endoscopy for widespread clinical use, such as in colorectal cancer screening. Moreover, sufficiently high production cost might also prohibit disposal of the robotic endoscope after each use. As disposal would not be generally practical according to these prior approaches, sterilization of the robotic endoscope becomes a likely necessity. Furthermore, sterilizing such a complex device with multiple mechanical and electronic components would be still a further challenge of substantial difficulty. The difficulty in sterilizing these robotic endoscopes could result in elevated potential for infectious disease transmission.

Other medical devices have also been previously disclosed that operate, at least in part, in much the same fashion as the robotic endoscopes just described. At least one additional medical device has been disclosed that uses an expandable front and rear cuff section with an expandable center section to produce movement, sharing certain similarities, including various of the incumbent shortcomings and concerns, with the robotic endoscope noted above. Another lumen-traversing device has also been disclosed that also shares certain similar limitations as the robotic endoscopes noted.

The disclosures of the following issued U.S. patents are herein incorporated in their entireties by reference thereto: U.S. Pat. No. 4,117,847 to Clayton; U.S. Pat. No. 4,207,872 to Meiri et al.; U.S. Pat. No. 4,321,915 to Leighton et al.; U.S. Pat. No. 4,368,739 to Nelson, Jr.; U.S. Pat. No. 4,561,427 to Takada; U.S. Pat. No. 4,615,331 to Kramann; U.S. Pat. No. 4,676,228 to Krasner et al.; U.S. Pat. No. 4,776,845 to Davis; U.S. Pat. No. 5,236,423 to Mix et al.; U.S. Pat. No. 5,259,364 to Bob et al.; U.S. Pat. No. 5,331,975 to Bonutti; U.S. Pat. No. 5,337,732 to Grundfest et al.; U.S. Pat. No. 5,398,670 to Ortiz et al.; U.S. Pat. No. 5,562,601 to Takada; U.S. Pat. No. 5,586,968 to Grundl et al.; U.S. Pat. No. 5,662,587 to Grundfest et al.; U.S. Pat. No. 6,071,234 to Takada; U.S. Pat. No. 6,086,603 to Termin et al.; and U.S. Pat. No. 6,224,544 to Takada. The following U.S. Patent Application Publications are also herein incorporated in their entireties by reference thereto: US 2002/0143237 to Oneda et al.; US 2003/0225433 to Nakao; US 2004/0106976 to Bailey et al.; and US 2004/0138689 to Bonutti.

### SUMMARY OF THE INVENTION

Although numerous approaches to developing and implementing endoscopic devices and methods, particularly for colon screening, have been proposed, there is still a need for improved endoscope delivery, in particular relation to colonoscopy. There is, in particular, still a need for an improved system and method that actively propels endoscopes within tortuous body lumens, and in particular the colon and lower GI tract, with improved control and substantially reduced wall trauma and pain. There is also still a need for an improved system and method that modifies commercially available endoscopes for active propulsion along body lumens.

This present invention provides a system and method adapted to assist movement of devices through body spaces, and in particular body lumens. In exemplary embodiments, it provides a system and method to assist endoscope movement along body spaces, such as lumens. For example, in some embodiments, it provides a system and method to assist movement of devices, and in particular endoscopes, through the colon and lower gastrointestinal tract.

One advantage provided by the present invention is a safe and effective low cost method for colon cancer screening. To achieve this end, the invention provides an endoscopic propulsion unit that can attach to currently available colonoscopes. The endoscopic propulsion unit can advance a colonoscope in the colon lumen without stretching the colon wall, greatly reducing procedure-related pain. An additional advantage provided by the invention relates to safety. For example, safety of colonoscopy is improved through the use of the present invention by reducing or eliminating the risk of colon perforation. In contrast to other propulsion units, the endoscopic propulsion unit of the present invention advances a colonoscope by pulling the distal end of the instrument. This reduces the likelihood of perforations, and reduces the amount of pain experienced by the patient. Furthermore, the present invention allows relatively painless colonoscopy that can be performed safely in a physician's office. By removing the need for high level sedation, colonoscopy can now be moved to a lower cost center, such as a physician's office or outpatient clinic. This movement away from hospital settings could result in a 66% or greater savings in the total colonoscopy cost. This comfortable, effective, affordable and safe method for colon cancer screening provided by the present invention can be widely used to reduce colon cancer mortality. Other advantages will be realized through consideration of the following disclosure and practice of the invention.

In a first aspect, the invention provides a device, such as one for use with a medical instrument. The device is capable of self-propelled motion through cavities defined by one or more walls, such as pipes and tubes, and such as body spaces, cavities, lumens, etc. (used interchangeably herein to denote an area within an animal, including human, body that is defined and bordered by a wall). When attached to another instrument, such as a medical instrument, provides the instrument with the ability to move through the cavities, such as body spaces, substantially without propulsive force provided by a human, or with relatively little human force. In general, the device comprises a drive unit or transmission for converting rotational energy from a drive shaft into longitudinal (i.e., forward or backward) movement of the device along a cavity. The drive unit comprises means for receiving one or more drive shafts, such as a drive shaft receptacle; means for converting rotational force provided by the drive shaft to longitudinal force, such as a radial gear, a series of interconnecting gears, a worm gear, or other functional drive, such as a friction drive, a magnetic drive, or direct gear drive; means for providing the longitudinal force of the drive unit to an exterior surface of the drive unit to cause the drive unit to move longitudinally, such as a rotatable rod or band comprising a suitable surface; and means for translating the longitudinal force of the drive unit to longitudinal force exerted against a cavity surface to cause the drive unit to move longitudinally along the cavity, such as a membranous element comprising a surface that releasably contacts the means for providing longitudinal force to a surface of the drive unit and releasably contacts the cavity surface, or a membranous element in combination with other structure to provide a surface or surfaces capable of such releasably contacting, such as a balloon in combination with belts. As can be seen, the device of the invention comprises two sub-parts that can be provided separately but combined to function together. That is, the drive unit may be provided with or without the means for translating longitudinal force from the drive unit to the cavity surface; where the two are provided separately, they can be combined to provide a unitary device.

In embodiments where the drive unit is adapted to connect to another instrument, such as a medical instrument, for example an endoscope, the drive unit comprises means for connecting to the instrument, such as a support tube traversing the length of the drive unit, typically located in the center of the drive unit when viewed on cross-section from one end or the other. Furthermore, the drive unit can comprise means for assisting in the attachment and release of the means for translating force from the drive unit to the body cavity surface, such as one or more support assemblies that can support a membranous element and guide it during attachment and/or release from the drive unit.

In embodiments, drive units for propulsion devices according to the present invention can comprise: (a) a functional drive, such as a worm drive, a friction drive, a magnetic drive, or a direct gear drive; (b) one or more flexible belts capable of engaging with the worm drive, such as a belt with tread; and (c) a membranous element circumscribed by the belts (e.g., an annular balloon or an annular invaginating balloon), wherein the worm drive is capable of translating rotational energy generated by a drive shaft to rotational movement of the belts around and/or with the balloon, which (particularly when inflated) supports the belt(s) and imposes pressure on the belt(s) for engaging the inner surface of a cavity (e.g., the wall of a gastrointestinal tract), thereby converting the rotational movement of the belts to longitudinal movement of the device through the cavity.

In a second aspect, the invention provides an article of manufacture for use with a drive unit of the invention, and preferably with another instrument, such as a medical instrument. The article provides the instrument with the ability to move through cavities, such as body spaces, and thus can be a means for translating longitudinal force from the drive unit to the cavity surface. In general, the article comprises a membrane that is generally toroidal in shape, having a single surface defining an inner surface, an outer surface, and front and back surfaces, all defined with respect to a mechanical device in conjunction with which the article is used. The article of manufacture of this aspect of the invention finds particular use in combination with the drive unit described herein. However, it may find uses in other devices, including medical devices in which self-movement of medical equipment (e.g., colonoscopes) through body cavities is desired. Indeed, when used in combination with the device of the first aspect of the invention, the article of manufacture of this aspect of the invention is particularly well suited for use in many fields, including, but not limited to engineering, fluid transfer technologies (e.g., inspection/repair of underground pipes, fuel lines, aircraft or other internal combustion engine-driven machinery parts), and medical (e.g., endoscopy). In general, in embodiments, the article of manufacture is fabricated in conjunction with a medical device, and thus its size, general shape, and composition can vary. However, in general, it is limited in size by its use in medical equipment and in its shape and fabrication by its function in the context of medical equipment for use inside a human or animal body cavity. Where used in non-medical settings, the size will be dependent on the size of the cavity, tube, line, pipe, etc. in which the device is to be used.

In a third aspect, the invention provides a medical device for performing diagnostics or surgery. The medical device according to this aspect of the invention comprises a combination of the device of the first aspect of the invention and the article of manufacture of the second aspect of the invention. The medical device is capable of traveling longitudinally along a body space defined by a wall using a propulsion mechanism that does not rely on human strength. It is thus a self-propelled medical device for traversing body cavities.

In another aspect, the invention provides an endoscope comprising an element that permits the endoscope to travel longitudinally using a propulsion mechanism, which is not force provided by human strength. The endoscope generally comprises a standard endoscope unit to which is attached, either fixedly or removable, a self-propelled device comprising a drive unit that is functionally linked to a membranous element. The endoscope is capable of self-propulsion through a body cavity through the action of the self-propelled device, which couples rotational movement of a drive shaft to backward and/or forward movement of the device by way of linkage of the drive shaft to the membranous element.

In a further aspect, the invention provides an endoscope comprising one or more drive shafts for connection to a drive unit that provides self-propelled movement through a body cavity. The drive shaft(s) are physically connected to the endoscope and a means for controlling movement of the endoscope when physically attached to a drive unit of the invention, such as an external drive unit and/or speed controller. In some embodiments, the endoscope further comprises one or more means for coupling the endoscope to a drive unit, such as one or more collars that releasably connect a drive unit to the endoscope.

In yet another aspect, the invention provides a method of diagnosis of a disease or disorder. In general, the method comprises inserting a device according to the present invention into a body cavity of a subject, and determining if one or more symptoms of a disease or disorder is evident in that body cavity. In certain embodiments, the method further comprises moving the device, via self-propulsion, longitudinally through the body cavity to observe some, most, or all or essentially all of the body cavity, or to otherwise determine if one or more symptoms of a disease or disorder exists. In exemplary embodiments, the method is a method of visualizing one or more abnormal growths in or on the surface of a body cavity.

In a further aspect, the invention provides a method of treatment of a disease or disorder. In general, the method comprises inserting a device according to the present invention into a body cavity of a subject, determining if one or more symptoms of a disease or disorder is evident in that body cavity, and, if one or more symptoms exist, treating the symptom(s). In certain embodiments, the method further comprises moving the device, via self-propulsion, longitudinally through the body cavity to observe some, most, or all or essentially all of the body cavity, or to otherwise determine if one or more symptoms of a disease or disorder exists. In exemplary embodiments, the method is a method of using an endoscope, such as a colonoscope, comprising the drive unit of the invention to identify one or more abnormal growths, such as polyps in or on the surface of a body cavity, such as the colon, and removing the abnormal growths.

Other aspects provide use of the devices, instruments, and articles in diagnosis and treatment of one or more diseases and/or disorders. The uses may be clinical and therapeutic. The uses may be experimental. The uses may be prophylactic, such as when a non-cancerous growth is removed from a body cavity under situations where it is known that the presence of the non-cancerous growth is highly correlated with a later development of a cancerous growth, such as in the case of polyps that are present in a colon. In yet other aspects, the invention provides for use of the devices, instruments, and articles in industrial and non-medical fields. The uses may be diagnostic, for example to determine if a fuel line is blocked or fractured, or may be reconstructive, for example by clearing a blocked line or pipe to restore function to it.

### BRIEF DESCRIPTION OF THE DRAWINGS

The accompanying drawings, which are incorporated in and constitute a part of this specification, illustrate several embodiments of the invention, and together with the written description, serve to explain certain principles of the invention:

FIG. 1 shows a perspective view of one embodiment of an endoscope and propulsion system (drive unit) according to a preferred embodiment of the invention;

FIG. 2 shows a perspective view of one embodiment of a propulsion system according to the invention, in particular, the device of FIG. 1 without the endoscope;

FIG. 3 shows a perspective view of one embodiment of an endoscope and propulsion system according to the invention, in particular, the device according to FIG. 1 with some components removed to view more internal components of the device;

FIG. 4 shows a perspective view of one embodiment of an endoscope and propulsion system according to the invention, in particular, the device according to FIGS. 1 and 3 with components removed to view other components of the device;

FIG. 5A shows a perspective view of one embodiment of the housing for a portion of the drive unit;

FIG. 5B shows a perspective view of one embodiment of a portion of the drive unit, including the drive shaft, drive gears, functional drive, and housing;

FIG. 5C shows a perspective view of one embodiment of a portion of the drive unit, including the drive shaft and gears, the functional drive, and housing;

FIG. 5D shows a perspective view of one embodiment of the functional drive and support;

FIG. 6 shows a side view of one embodiment of a portion of the drive unit, including the drive shaft and gears, the functional drive, and flexible belts;

FIGS. 7A and 7B show, respectively, a top and side view of one embodiment of a belt;

FIG. 8 shows a cross-sectional view of one embodiment of a portion of the drive unit, including the functional drive and gears, belts, and balloon;

FIG. 9 shows a cross-sectional view of one embodiment of a portion of the drive unit, including the drive shaft, support for the functional drive, and belts;

FIG. 10 shows a side view of one embodiment of a portion of the drive unit, including the drive shaft, the functional drive, and flexible belts;

FIG. 11 shows a perspective view of a portion of the drive unit, including the cowling, drive shaft, and supports for the functional drive;

FIG. 12 shows a perspective view of one embodiment of a cowling;

FIG. 13 shows a cross-sectional view of one embodiment of a portion of the drive unit, including the proximal support, the balloon, and flexible belts;

FIG. 14 shows a perspective view of one embodiment of a propulsion system according to the invention, in particular, showing inflation of the balloon;

FIG. 15 is a perspective view showing the gearing;

FIG. 16 is a perspective view showing the assembly; and

FIG. 17 is a perspective view showing the assembly except with the casing rendered transparent.

### DETAILED DESCRIPTION OF VARIOUS EMBODIMENTS OF THE INVENTION

Reference will now be made in detail to various exemplary embodiments of the invention, examples of which are illustrated in the accompanying drawings. The following detailed description is provided to detail various elements, combinations, and embodiments of the invention, and is not intended as a limitation of the invention to the particular elements, combinations, and embodiments exemplified.

In a first aspect, the invention provides a device, such as a medical device or a device for use in non-medical situations. The device may be used for diagnostic purposes and therapeutic purposes in its medical embodiments, and for diagnostic and reparative purposes in its non-medical embodiments. In its various embodiments, it is appropriately sized to fit and function within the particular cavity it is to be used in. Thus, in embodiments, it is sized to fit in a human cavity, such as a colon, vein, or the like. It also may be used in conjunction with another instrument of device, such as a medical device or medical instrument for diagnostics, repair, and/or therapeutics. The device of the invention is capable of self-propelled motion through cavities, such as body cavities, using little or no propulsive force provided by a human. When attached to a separate instrument, such as a medical instrument, the device of the invention provides the instrument with the ability to move through cavities, such as pipes, tunnels, tubes, and body spaces, substantially without propulsive force provided by a human.

In general, the device of the invention comprises a drive unit or transmission for converting rotational energy from a drive shaft into longitudinal (i.e., forward or backward) movement of the device along a cavity, such as a body space. The drive unit comprises means for receiving one or more drive shafts; optional means for converting rotational force provided by the drive shaft to longitudinal force; means for providing the longitudinal force of the drive unit to an exterior surface of the drive unit to enable the drive unit to move longitudinally; and means for translating the longitudinal force of the drive unit to longitudinal force exerted against a cavity surface to cause the drive unit to move longitudinally along the cavity.

According to the present invention, the means for receiving one or more drive shafts can be any suitable structure that permits an externally provided force to be converted to an internal force of the drive unit. It is often a physical element capable of providing rotational force to deliver that force to the drive unit of the invention. However, it can be air or other fluid pressure. While not so limited in structure or function, typically, the physical element that provides rotational force (referred to generally herein as a drive shaft) will be a wire, flexible rod, cable, or the like, which is connected on one end to a source of rotational energy and connected on the other end to the drive unit. While not necessary, typically the drive shaft will be encased in a protective sheath or coating, which will not rotate as the drive shaft rotates, to protect it and biological tissue or the like that it might contact from damage. Examples of means for receiving one or more drive shafts or the like include, but are not limited to, recesses or holes in an end support or collar of the drive unit, provided either within the general structure of the support or collar or as an additional element attached to a support or collar. Other examples include, but are not limited to, flanges or brackets attached to the drive unit, preferably at or near one end, but not necessarily so limited in placement. Thus, in embodiments, a drive shaft enters the drive unit through a hole in a surface of the drive unit.

According to the invention, the means for converting and external force (e.g., rotational force provided by a drive shaft) to longitudinal force can be any suitable element that is capable of converting the forces from one to the other. Non-limiting examples are one or more gears, cogs, sprockets, etc., or combinations of two or more of these in functional and physical contact. Various configurations of gears and the like are known in the art, and any suitable configuration is envisioned by the present invention. In exemplary embodiments, the means comprises at least one gear. In other exemplary embodiments, the means comprises a radial gear. Where desired, the drive unit may also comprise means for connecting a means for providing rotational force (e.g., drive shaft) to means for converting rotational force to longitudinal force. Thus, in embodiments, a drive shaft enters the drive unit through a hole in a surface of the drive unit; the drive shaft is physically connected to a first gear; and the gear is physically connected to a second gear, which causes a functional gear that traverses the length or essentially the length of the drive unit to turn.

According to the invention, the means for providing the longitudinal force of the drive unit to an exterior surface of the drive unit to enable the drive unit to move longitudinally can be any suitable physical element or combinations of elements. Non-limiting examples are functional gears that rotate along the long axis of the drive unit and have a surface that comprises one or more projections or troughs that spiral about the outer surface from one end to the other. Other non-limiting examples are bands or sheets of flexible material (e.g., rubber or other elastic material, nylon, cloth) that can be driven by gears to rotate longitudinally along a surface of the drive unit, similar to a treadmill tread, an escalator tread, or a moving sidewalk). The bands or sheets may be designed to comprise an outer surface that interacts with another complementary surface. For example, a flexible plastic band may comprise an outer surface that comprises hooks for mating with loops that are present on an outer surface of a means for translating longitudinal force to a body cavity surface. Alternatively, it may comprise a wave pattern that is complementary to a wave pattern on a means for translating longitudinal force to a body cavity surface. Additionally, it may comprise any number of other surface geometries and patters that cause it to releasably attach to a complementary surface of a means for translating longitudinal force to a body cavity. Any number of materials and geometries may be envisioned by those of skill in the art, and all suitable materials, geometries, and combinations are encompassed by the present invention. Thus, in embodiments, a drive shaft enters the drive unit through a hole in a surface of the drive unit; the drive shaft is physically connected to a first gear; and the gear is physically connected to a second gear, which causes a functional gear that traverses the length or essentially the length of the drive unit to turn. Turning of the functional gear causes projections on the surface of the gear, which are disposed on the surface in a manner to create spirals running from one end to the other, to rotate, providing longitudinal force for movement of the drive unit along a body cavity.

The drive unit of the invention may, in embodiments, comprise means for translating the longitudinal force of the drive unit to longitudinal force exerted against a cavity surface, such as a pipe or body cavity, to cause the drive unit to move longitudinally along the cavity. While the means can take any physical form, typically, the means will comprise a flexible material that can releasably attach to both the drive unit and a surface of a cavity. In essence, the means functions as a tread connecting the drive unit to the cavity surface. Non-limiting examples of this means include flexible balloon-like structures that can be provided in a small, deflated state, then inflated to obtain a larger, functional state. The surface of the means is preferably designed to be complementary or otherwise capable of attachment to the means for providing the longitudinal force of the drive unit to an exterior surface of the drive unit to enable the drive unit to move longitudinally. Accordingly, the surface may comprise loops, for use in a hook-and-loop combination. It likewise may comprise projections or troughs to accommodate troughs or projections on a complementary surface on the surface of the drive unit. It further may comprise any geometry or surface feature or characteristic that permits successful releasable attachment to a surface of interest, and in particular to a surface on the drive unit and to a surface on a cavity, such as a biological cavity. Thus, in embodiments, a drive shaft enters the drive unit through a hole in a surface of the drive unit; the drive shaft is physically connected to a first gear; and the gear is physically connected to a second gear, which causes a functional gear that traverses the length or essentially the length of the drive unit to turn. Turning of the functional gear causes troughs on the surface of the gear, which are disposed on the surface in a manner to create spirals running from one end to the other, to rotate. Projections on the surface of a membranous element, which are complementary to the troughs on the surface of the functional gear, engage the functional gear along the length of the drive unit. As the functional gear turns, projections at the rear of the gear are moved forward. This movement is translated to movement of the entire membranous element, part of which is releasably attached to a cavity surface, providing longitudinal force for movement of the drive unit along the cavity.

Likewise, means for translating the longitudinal force of the drive unit to longitudinal force exerted against a cavity surface can comprise a combination of more than one flexible material that cooperate together to releasably attach to both the drive unit and a surface of a cavity. For example, one or more flexible belts (e.g., with tread) can circumscribe and cooperate with a membranous element (e.g., an annular balloon or an annular invaginating balloon) to provide such means. In such an embodiment, the belts are configured to be capable of engaging the functional gear and the cavity surface to propel the device longitudinally through the cavity upon rotation of the functional gear, such as belts comprising tread. The cooperating membranous element circumscribed by the belts is capable of providing support for the belts and capable of providing pressure on the belts to facilitate engagement of the belts with the surface of the cavity. For example, the membranous element can be inflated or deflated (e.g., with fluid) to control the engagement pressure between the belts and the cavity wall, for example, to accommodate traversal of the device through different size cavities. Further, the membranous element can be fixed, while the belt(s) traverse the membranous element during longitudinal movement of the device through or within the cavity. Or the surface of the membranous element can move in the same direction as the belt(s) and add to the engagement function of the belt with respect to the cavity surface (e.g., by way of an annular invaginating balloon).

In certain embodiments, the drive unit comprises means for separating the means for providing longitudinal force to the drive unit and the means for translating the longitudinal force of the drive unit to longitudinal force exerted against a surface, such as a body cavity surface. The separating means may comprise any physical element that provides the stated function. Thus, it may be a simple physical separator positioned to split the two means from each other at an end of the drive unit. The shape and material of fabrication are not critical in providing the separating means. Thus, it may be any number of sizes, shapes, and materials. In embodiments, it is a flat plate connected to an end support or collar of the drive unit, positioned such that it lies in or near the plane of contact between the surfaces of a hook-and-loop complementary pair, one surface on the drive unit and the other on a membranous element that contacts the drive unit and the surface of a cavity. Thus, the separating means may be fabricated from one or more metals, metal alloys, plastics, and the like, and combinations of two or more of these. Those of skill in the art are well aware of suitable materials, shapes, and sizes to provide the separator function.

Furthermore, the drive unit can comprise means for assisting in the attachment and release of the means for translating force from the drive unit to the cavity surface, such as one or more support assemblies that can support a membranous element and guide it during attachment and/or release from the drive unit. This means may comprise the means for separating, as described above.

It should be evident that, in embodiments, the drive unit may comprise means for providing a force to the drive unit. In embodiments, the force is a rotational force. In other embodiments, the force is a longitudinal force, such as that provided by a fluid pressure, such as air pressure. The means may be any suitable physical element, including, but not limited to, a drive shaft, rod, wire, cable, or the like. For ease of reference, this element is referred to herein as a drive shaft. In embodiments, the device comprises multiple (e.g., two, three, four, five, or more) drive shafts, and, preferably, an equivalent number of means for accommodating them and functionally coupling them to the drive unit. It is to be noted that the use of two or more provides stability and control of the unit as it traverses cavities, such as biological cavities and man-made cavities. It is further to be noted that the use of three or more provides three-dimensional steering to the device, allowing the practitioner to guide the device in multiple directions within a cavity. Clearly, the more drive shafts and/or independently controllable means for providing the longitudinal force of the drive unit to an exterior surface of the drive unit to enable the drive unit to move longitudinally will provide increasing control over the device.

As can be seen, the device of the invention comprises at least two sub-parts that can be provided separately but combined to function together. That is, the drive unit may be provided with or without the means for translating longitudinal force from the drive unit to the cavity surface. Where the two are provided separately, they can be combined to provide a unitary device.

In some embodiments, the device is designed to be an autonomous unit for, among other things, diagnosis of a disease or disorder in an animal or human body, and diagnosis and optional repair of a man-made conduit, such as a tube, pipe, line, etc. Various examples of such embodiments are described above. In other embodiments, the device is designed to be used in conjunction with another device, for example a medical device, such as an endoscope. In such embodiments, it may be used, among other things, for diagnosis and treatment. In embodiments where the drive unit is adapted to connect to an instrument, such as a medical instrument, such as an endoscope, the drive unit comprises means for connecting it to the instrument. In general, the means will be some sort of indentation, invagination, cavity, or hole in the drive unit. In exemplary embodiments, the means is a hole traversing the longitudinal length of the drive unit. In particular embodiments, this through hole is referred to as a support tube, which, like other embodiments, comprises an inner and outer surface defining a cavity or space into which or through which another device, or a part thereof, may be disposed, either removably or permanently. The element may be fabricated in any shape and from any material. Typically, the means will traverse the length of the drive unit, and will typically be located in the center of the drive unit when viewed on cross-section from one end or the other.

As mentioned above, the invention provides a device for use with a medical instrument. The device provides the ability to move through body spaces in a human or animal without significant or essentially any force provided directly by a human. The device is thus a self-propelled drive unit that can be used in conjunction with other medical devices or instruments and with one or more articles of manufacture to provide diagnostic and/or therapeutic treatments to subjects.

In view of its usefulness in conjunction with other devices or instruments, such as medical devices or instruments, in embodiments the device of the invention comprises a drive unit or transmission for converting rotational energy or force from a drive shaft into forward and/or backward movement of the device along a cavity, such as a tube or body space. In these embodiments, the drive unit comprises a support tube traversing the length of the drive unit and typically, but not always, located in the center of the drive unit when viewed on cross-section from one end or the other. While the support tube may provide numerous functions, in many embodiments, it serves as a conduit for a tube, such as an endoscope tube. As with all other elements of the device and article of manufacture of the present invention, the support tube may be fabricated out of any suitable material, including, but not limited to, plastics, polymeric, elastomeric, or other synthetic rigid, semi-rigid, or flexible materials; metals or metal alloys, such as steel, stainless steel, and aluminum; and composite materials, such as fiberglass and carbon composites; and the like. The selection of any particular material may be made by the practitioner without undue experimentation based on numerous considerations that are typical in the field, such as, but not limited to, size, cost, need for flexibility, whether the unit will be disposable or reusable, weight, availability of materials, and the like. Furthermore, while exemplary embodiments depict the support tube as having a round cross-section, it is to understood that the cross-section can take any shape, including, but not limited to, round, oval, elliptical, square, rectangular, hexagonal, octagonal, trapezoidal, and polygonal. The choice of shape may be made in consideration of many factors, including shape of the instrument to which the device will be connected, ease of manufacture, etc.

In addition to the support tube, the drive unit may further comprise one or more support assemblies, typically with one located at one or each end of the drive unit and attached to the support tube or the drive unit body. As with all elements that are attached to other elements, unless specifically noted otherwise for a particular embodiment, the support assemblies are attached to the support tube or drive unit body in any suitable fashion. Thus, they can be permanently (i.e., fixedly) attached, for example by way of chemical or mechanical fusion or welding; adhering, such as through the use of glue or other adhesives; or by use of any other type of permanent fastening means. Alternatively, they can be removably attached, for example, by way of one or more removable mechanical fasteners, such as by pinning; bolting; screwing; stapling; riveting; friction fitting; or by use of any other type of removable or reversible fastening means.

In some embodiments, each support assembly comprises or defines a hole that is identical or substantially similar in cross-sectional shape to the shape of the hole defined by the support tube. In their basic form, each support assembly comprises an end support comprising or forming the hole. The end support can take any shape, but is typically fashioned to comprise at least one exterior surface that faces away from the device, a mating surface that physically contacts the support tube or drive unit body, and at least one interior surface, which faces toward at least one other element of the device and which may be designed to comprise, at least over a portion of its length, a shape that guides moving elements of the device or devices or instruments for which it is a part. For example, where the interior face contacts a membranous element that functions in movement of the device along a body cavity wall, the interior face may be shaped in such a way as to receive the membranous element as it detaches from the cavity wall, and guide the membranous element toward one or more drive wheels, which contact the membranous element and cause it to move.

As should be evident, in some embodiments, the end support is designed to function in conjunction with a membranous element that contacts both the device and the wall of the body cavity in which the device is inserted. In these embodiments, the end support can have a height that varies according to the height of the membranous element. For example, in some embodiments, it has a height that approximates one-half or less of the height of the membranous element, from the point of contact of the membranous element with the device at the point closest to the support tube to the point of contact of the membranous element with the body cavity wall. In other embodiments, the end support extends one-half or more of the height. In certain embodiments, the end support extends at least about two-thirds (67%), three-fourths (75%), or four-fifths (80%) of the height. In other embodiments, it extends at least about 85%, 90%, 95%, 97%, or 99% of the height. In some embodiments, the end support extends greater than 99% of the height, such that it might make contact with the body cavity wall at certain times or continually during use of the device. The height of extension can be selected based on any number of considerations, including, but not limited to, the propensity of the membrane to adhere to the cavity wall, the composition and surface structure (e.g., smoothness, roughness) of the interior surface of the end support, and the composition and surface structure of the membranous element. Of course, in some embodiments, one, some, or all of the end supports are omitted.

Where the end support is used as a guide for the membranous element onto one or more drive mechanisms of the device (for example, an outer drive wheel), the interior surface may be generally curved from top to bottom, providing a curving ramp-like structure that guides the membranous element onto the drive mechanism(s). While an end support that does not guide the membranous element onto the drive mechanisms is envisioned, for obvious reasons, it is preferred that the end supports be shaped to provide at least some guidance for the membranous element.

It is to be noted that each end support may be designed independently of the other. Thus, in any one drive unit, multiple different end support, and thus multiple different support assemblies, may be present.

The support assembly may further comprise one or more outer drive wheels, which may be directly attached to the end support, the support tube, or both. Alternatively, each drive wheel may be independently attached via a mating groove to the support tube. In embodiments, the drive unit comprises two outer drive wheels, one located on each end of the unit, and attached as part of a support assembly, respectively. While not so limited, these outer drive wheels may function in conjunction with the end support to capture and move an attached membranous element, to assist in movement of the device along a cavity.

In some embodiments, one or more outer drive wheels are connected, physically and functionally, to a drive shaft via an intermediate drive wheel. The intermediate drive wheel may be physically connected to the support tube by way of a mating groove.

Within the drive unit, there also may be disposed one or more inner drive wheels. The inner drive wheel(s) can be provided to couple the rotational force of a drive shaft to the longitudinal force created by the intermediate drive wheel(s) and outer drive wheel(s). Thus, the inner drive wheels are physically connected to the intermediate drive wheels and to a drive shaft.

In view of the function of the device, in embodiments the drive unit comprises a drive shaft, which connects the drive unit to a power unit, which is typically located outside of the cavity in which the device is inserted and used. Although numerous configurations are possible, in a typical configuration, the drive shaft is connected to at least one inner drive wheel, serving as the axle for the wheel. As the drive shaft is connected to the inner drive wheel, and as this wheel is typically located within the interior spaces of the drive unit, in a typical configuration, the drive unit comprises a conduit, tube, through-hole, etc. to accommodate the drive shaft, which may or may not be encased in a protective sheath to isolate the rotational movement of the shaft from other elements of the device and from other materials, such as biological tissues. The through-hole may be disposed within the drive unit in any position, as long as the drive shaft is able to connect from the power unit to at least one inner drive wheel.

Within the drive unit, multiple outer drive wheels may be provided. Each may be provided associated with intermediate and inner drive wheels. Each may be disposed along the length of the support tube at any position. Exemplary embodiments depicted in the drawings show the presence of two outer drive wheels; however, it is to be understood that three or more wheels may be provided, for example to provide more support for a membranous element, to provide higher surface area for attachment of the device to a membranous element, or any other reason. Where multiple drive wheels are used, the height of each wheel, with respect to the support tube, may be selected independently to achieve any particular goal. For example, where a relatively tall toroidal shaped membrane is used, two end outer drive wheels may be provided, one at each end, and one central outer drive wheel may be provided. The two end outer drive wheels may be relatively tall with respect to the central outer drive wheel to ensure suitable contact with the membrane element as it traverses down one side of the drive unit to the bottom (at or near the central outer drive wheel) and then back up the other side.

The invention thus provides a device having means for driving, in a self-propelled manner, itself and other medical equipment and devices attached to it, through a cavity, such as a body cavity. The device comprises means for supporting one or more drive elements, which may also be a means for providing a through-passage for one or more elements of a medical instrument, such as an endoscope. Such a means may also simply be a structural framework or body for the device, fabricated in any suitable shape and of any suitable material. The device of the invention further has means for supporting an element that contacts the device and the wall of a cavity, which means may also provide guidance to the element as it enters and/or leaves the device. One or more means for driving the element across the length of the device are also provided.

As used herein, a subject or patient is a human or animal for whom medical treatment is intended. The subject can be any age or sex, and can show no, one, or multiple clinical signs of a disease or disorder. If an animal, the subject can be any animal, but will typically be one of commercial, medical, or scientific value, such as a farm animal, a companion animal, or a research animal. Non-limiting examples of animals include: dogs, cats, horses, cattle, sheep, pigs, rodents (e.g., rats, mice), and wild animals in captivity (e.g., elephants, tigers or other wild cats, monkeys, apes). Thus, the invention has applicability to both the human and veterinarian medical fields.

In a second aspect, the invention provides an article of manufacture for use with an instrument, such as a medical instrument. The article provides the instrument with the ability to move through cavities, such as body spaces, tubes, lines, pipes, and the like. In general, the article comprises a membrane (also referred to herein as a membranous element) that is toroidal in shape, having a single unitary surface defining an inner surface, an outer surface, and front and back surfaces, all defined with respect to a mechanical device in conjunction with which the article is used. The article may be air and/or water tight, and may be inflatable and deflatable. In this way, the article may be positioned within a cavity, inflated to create a contact with the cavity wall for use, then deflated for ease of removal upon completion of the desired task.

As used herein, the term “membrane” means any material that can be formed into a toroidal shape of a suitable size, strength, and flexibility to be used in conjunction with a drive unit according to the invention. It thus may be made from any material that can be provided in a thin sheet suitable for flexing about three dimensions without crimping, folding, cracking, or breaking. Suitable materials for such applications are known in the art and include, without limitation, materials such as or comprising latex or other natural or synthetic rubbers, nylon, polymeric materials, plastics, and fabrics (with man-made and/or natural fibers). As a general matter, preferred membranes have relatively low coefficients of friction with the interior surface of the end supports of the drive unit of the invention, but sufficient coefficients of friction with materials from which cavity walls are fabricated, such as, in the case of biological materials, walls of body cavities. In this way, the membrane slides relatively easily over the end supports of the device while adhering relatively strongly to the cavity wall, thus promoting movement of the device across and along the cavity. It is also preferred that the membrane have a sufficient coefficient of friction with regard to the drive wheels, again promoting movement of the device. Additionally, a sufficient coefficient of friction of the drive unit with respect to the cavity walls may be achieved by using one or more belts (e.g., belts with tread) in combination with the membranous element. One of skill in the art will recognize that different types of tread will impart different coefficients of friction, which may be useful for different applications. Further, one of skill in the art will recognize that belts comprising certain materials may need no tread if such materials have characteristics contributing to the desired amount of friction. Suitable tread provides a force sufficient to provide mobility of the device with little or no detrimental impact on the lumen, which can be incorporated into the flexible belts and/or the membranous element. As with all other components of the invention, as broadly described herein, preferably, the membrane is comprised of substances that can be sterilized by one or more means, such as by heat (e.g., autoclaving) or irradiation. In addition, as with all other components of the invention, in some embodiments, the membrane is sterile or has been sterilized.

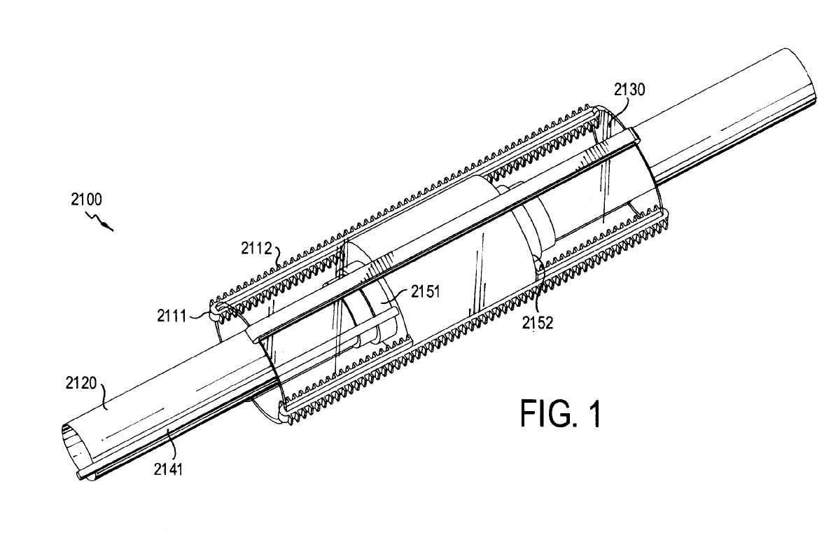

The membrane may be fabricated in any suitable shape. It thus may have a long, low profile, when viewed in cross-section along its long axis (see, for example, FIGS. 1, 2, 8, and 14). Alternatively, it may have a short, high profile, when viewed in cross-section along its long axis (for example, in a donut shape). The shape may be selected without undue experimentation based on any number of parameters, including, but not limited to, relative friction coefficients for body cavity walls and end support interior surfaces, total surface area desired to be in contact with cavity walls, etc. In addition to the overall three-dimensional shape of the membrane, the membrane may be fabricated with any number of surfaces. For example, the membrane may be fabricated with a smooth surface, a rough surface, or a surface comprising extensions, such as grooves, waves, bubbles, pins, spikes, rods, hooks, and loops, all of which can be aligned parallel to the line of motion, perpendicular to the line of motion, or randomly. Likewise, the individual characteristics (e.g., rough, wave, spike) can be used as the sole surface characteristic or in any combination, in any pattern (including random). The surface may be fabricated to advantageously interact or interconnect with the surface of one or more drive wheels of a drive unit of the present invention. Any modification to a smooth surface is contemplated by the present invention.

In embodiments where one or more belts (e.g., belts with tread) are used in combination with a membranous element to provide longitudinal movement of the device within the cavity, the surface of the belts (e.g., tread) can be adapted as just described to facilitate engagement of the belts with the cavity surface and/or with the drive unit. Suitable tread provides a force sufficient to provide mobility of the device with little or no detrimental impact on the lumen, which can be incorporated into the flexible belts and/or the membranous element. Further, one of skill in the art would recognize that no tread may be needed in certain applications if the belts comprise materials with characteristics that facilitate such engagement.

The membrane of this aspect of the invention finds particular use in medical devices, such as those used for movement of medical equipment (e.g., colonoscopes) through body cavities. When used in combination with the drive unit discussed above, the membrane is particularly well suited for use in endoscopy. It can be adapted to expand to fit any cavity of interest, providing good traction for the device without causing excessive extension of the body cavity, and producing associated pain.

In a third aspect, the invention provides a medical device for performing diagnostics or surgery. The medical device according to this aspect of the invention comprises the drive unit of the invention and, optionally, a combination of the drive unit and the membrane discussed above. According to the invention, the medical device is capable of traveling along a body space defined by a wall using a propulsion mechanism that does not rely directly on human strength. It is thus a self-propelled medical device for traversing body cavities. The medical device can advantageously be used, as compared to currently available technologies, as a self-propelled unit for diagnosis and/or therapy. In embodiments, it is used without connection to another device, such as an endoscope, and is used for diagnostic purposes only. In other embodiments, it is used in conjunction with a separate medical device, such as an endoscope, to provide diagnosis and/or treatment. The medical device is superior to similar devices in the field because it uses a gentle, self-propulsion mechanism to move the device (and any device connected to it) through a body cavity. When the device is connected to the distal end (i.e., tip) of a medical instrument, such as an endoscope, the movement caused by the device can be envisioned as pulling the device and instrument through the body cavity. This pulling action reduces the amount of pressure needed to move the device through the cavity, and reduces the likelihood of pain to the subject and perforation of the cavity wall due to excessive pressure being exerted to move a medical instrument through a body cavity. The device can be used without an endoscope and can have a steering mechanism that uses only the four belts to change direction as well as provide propulsion by selective movement of individual bands. In such a case, each band can be powered by its own drive cable with a viewing camera mounted on the drive unit that can be controlled either by wire or potentially wirelessly. Preferably, the medical device is produced to be disposable and is manufactured of inexpensive molded plastic parts. Alternatively, the medical device is sterile, has been sterilized, or is comprised of materials that can withstand one or more means of sterilization.

In other aspects, the invention provides a device for performing diagnostics or repair of man-made structures, such as pipes, lines, tubes, conduits, and the like. The device according to this aspect of the invention comprises the drive unit of the invention and, optionally, a combination of the drive unit and the membrane discussed above. According to the invention, the device is capable of traveling along a man-made space defined by at least one wall using a propulsion mechanism that does not rely directly on human strength. It is thus a self-propelled device for traversing man-made cavities. The device can advantageously be used, as compared to currently available technologies, as a self-propelled unit for diagnosis and/or repair of man-made cavities. For example, it may be used to diagnose and optionally repair fuel lines (including underground piping and pipelines) or other fluid-transporting lines. In embodiments, it is used without connection to another device, such as a boring or drilling device, and is used for diagnostic purposes only. In other embodiments, it is used in conjunction with a separate device, such as a drilling or patching device, to provide diagnosis and/or repair of a man-made cavity. The device utilizes a self-propulsion mechanism to move the device (and any device connected to it) through the cavity, and thus requires little or no external propulsive force to move it through the cavity. As with the medical embodiments of the invention, when the device is connected to the distal end (i.e., tip) of another instrument, the movement caused by the device can be envisioned as pulling the device and instrument through the cavity, a mode of movement that is highly efficient. This pulling action reduces the amount of pressure needed to move the device through the cavity, and reduces the likelihood of damage to the cavity or the device due to excessive pressure being exerted to move the instrument through the cavity.

In another aspect, the invention provides an endoscope comprising an element that permits the endoscope to travel longitudinally through a body cavity using a propulsion mechanism other than force provided by human strength. The endoscope generally comprises a standard endoscope unit to which is attached, either fixedly or removable, a self-propelled device comprising a drive unit that is functionally linked to a membranous element. The endoscope is capable of self-propulsion through a body cavity through the action of the self-propelled device, which, in exemplary embodiments couples rotational movement of a drive shaft to backward and/or forward movement of the device by way of linkage of the drive shaft to the membranous element, such as by way of linkage of the drive shaft to the membranous element by way of intermediate drive components, including a functional drive, such as a worm drive, and belt(s) that circumscribe the membranous element. Other examples of suitable functional drives include friction drives, magnetic drives, and direct gear drives. In embodiments, the endoscope comprises a camera or other means for visualizing the interior of the body cavity in which the endoscope is placed. In embodiments, the endoscope comprises surgical instruments or other means for performing surgery in the body cavity. In embodiments, the invention provides a colonoscope. In preferred aspects and embodiments comprising an endoscope, some or all of the device components or the endoscope in total is intended to be disposable and is manufactured using inexpensive plastic molded parts. Alternatively, the device is sterile, has been sterilized, or is capable of withstanding one or more sterilization techniques without losing function.

In a further aspect, the invention provides an endoscope comprising one or more drive shafts for connection to a drive unit that provides self-propelled movement through a body cavity. The drive shaft(s) are physically connected to the endoscope and a means for controlling movement of the endoscope when physically attached to a drive unit of the invention, such as an external drive unit and/or speed controller. In some embodiments, the endoscope further comprises one or more means for coupling the endoscope to a drive unit, such as one or more collars that releasably connect a drive unit to the endoscope.

In yet another aspect, the invention provides a method of diagnosis of a disease or disorder. In embodiments, it is also a method of diagnosing the likelihood of a subject becoming a sufferer of a disease or disorder. In general, the method comprises inserting a device or medical instrument according to the present invention into a body cavity of a subject, and determining if the subject is suffering from one or more diseases or disorders, or is at high risk of suffering from one or more diseases or disorders. The step of determining can be accomplished by identifying one or more symptoms of a disease or disorder in the body cavity. This can be done by visual observation of one or more symptoms, such as by visualization of one or more polyps on the colon wall of a patient, or by any other means that can provide the practitioner with a high level of confidence that a symptom exists.

In certain embodiments, the method further comprises moving the device, via self-propulsion or substantially by self-propulsion, through the body cavity to observe some, most, or all or essentially all of the body cavity, or to otherwise determine if one or more symptoms of a disease or disorder exists. In some embodiments, the device is attached to a medical instrument, such as an endoscope. In exemplary embodiments, the method is a method of using an endoscope, such as a colonoscope, to identify one or more abnormal growths in or on the surface of a body cavity. It is to be noted that the symptoms may be symptoms associated with a pre-disease state, which has a high correlation to a disease state. Accordingly, the invention may be a method of diagnosing a pre-condition for a disease, where the disease has not yet developed or is in a pre-clinical stage.

In a further aspect, the invention provides a method of treatment of a disease or disorder, or the treatment of a pre-clinical or pre-disease state of a patient. In general, the method comprises inserting a device or medical instrument according to the present invention into a body cavity of a subject, determining if one or more symptoms of a disease or disorder, or symptoms of a pre-clinical or pre-disease state, is evident in that body cavity, and, if one or more symptoms exist, treating the symptom(s) and/or the underlying cause(s) of the disease or disorder. In embodiments, the method further comprises treating the patient with one or more drugs or surgeries to reduce or eliminate the symptom(s) and/or underlying cause(s). Treatments may be repeated periodically as deemed advantageous by the practitioner or a medical consultant. Various treatment regimens for various diseases and disorders are known in the art and can be devised by medical practitioners without undue or excessive experimentation.

In certain embodiments, the method further comprises moving the device, via self-propulsion, through the body cavity to observe some, most, or all or essentially all of the body cavity, or to otherwise determine if one or more symptoms of a disease or disorder exists. In embodiments, the device is attached to a medical instrument, such as an endoscope. In exemplary embodiments, the method is a method of using an endoscope, such as a colonoscope, to identify one or more abnormal growths, such as polyps in or on the surface of a body cavity, such as the colon, and removing the abnormal growths.

Thus, one aspect of the present invention is a device and related method that is adapted to assist movement of a commercially available endoscope in an organ lumen. According to one mode, the device uses an external variable speed motor to provide torque. In one embodiment of this mode, an external control unit regulates rotational direction and speed. In a further embodiment, torque from the motor is transmitted to a flexible drive shaft that, according to one variation, runs through a slip coupling. In another further embodiment, the drive shaft is contained within a sheath that runs substantially along the length of the endoscope. In another further embodiment, the sheath is attached to the endoscope by brackets. In another further embodiment, the drive shaft is attached to an internal drive gear contained within a transmission.

In still a further transmission embodiment, the transmission comprises an internal drive gear, an intermediate gear, and an external drive gear, which are adapted to cooperate together, e.g., with various supports and couplings, necessary to allow for interaction and rotation of the individual gears. The internal drive gear turns an intermediate gear. According to one further feature, the intermediate gear may be held in position by bearing, which may include in one further embodiment a flexible tube. According to one variation of this feature, the flexible tube is coupled to the distal end of an endoscope, such as in one highly beneficial variation by attachment means that may include for example attachment brackets. Rotation of the intermediate drive gear causes rotation of external drive gears. The external drive gears are radially arrayed on the outside of the flexible tube. The external drive gears are in contact with the inner surface of an annular invaginating balloon. The annular invaginating balloon is donut shaped in cross-section with a length that may be adapted and varied in dimension to suit one or more particular applications. Interaction of the external drive gears with the annular invaginating balloon actuates rotation of the annular invaginating balloon along its long axis. The annular invaginating balloon is inflated after insertion into an organ lumen. This is accomplished in one particular variation by use of a cannula and a syringe. A sensor and/or indicator is provided that allows control of inflation to a desired parameter, such as for example pressure or volume. In one particular beneficial embodiment, a pressure sensor, which according to one variation may include a pressure-sensing bulb on the cannula, is adapted to allow control to an appropriate inflation pressure. After the annular invaginating balloon has been inflated to the appropriate pressure and/or other parameter such as volume, the cannula and pressure-sensing bulb (if provided) is removed. A valve, such as a self-sealing valve on the annular invaginating balloon, maintains pressure within the balloon. The annular invaginating balloon is in contact with the lumenal side of an organ wall. Interaction between the annular invaginating balloon and the lumenal wall produces dynamic rolling traction (like a tire or wheel). This rolling traction in turn moves the endoscope within the organ lumen. As an alternative to a syringe, an inflation pump can be used to instill air into the balloon. There is an inflation tube attached to the cowling that communicates with the balloon through an opening in the cowling. The preferred embodiment does not use a valve device, as noted in the above-cited previous patent.

Another aspect of the invention provides a delivery assembly that works in conjunction with endoscopes, such as for example currently available endoscopes. Another aspect of the current invention provides a delivery assembly that attaches easily to currently available endoscopes without generally requiring modification of such endoscopes. Another aspect of the current invention provides an endoscope delivery assembly that is easily used and requires minimal training of the endoscopist.

Another aspect of the current invention provides an endoscope delivery assembly with an annular invaginating balloon that is adapted to produce rolling traction along a luminal wall to move an endoscope in the lumen. According to one mode of this aspect, the invaginating balloon is adapted to be inflated with fluid to sufficiently low pressure such that trauma to the organ wall is substantially limited. According to another mode, the annular invaginating balloon has a sufficiently large surface area adapted to contact the luminal wall, thereby substantially limiting the required inflation pressure to provide traction along the wall and limiting the propensity for pressure-related trauma from the assembly. According to another mode, the annular invaginating balloon is provided as a modification to the endoscope, such as to currently available devices.

Another aspect of the invention provides an endoscope delivery assembly that is adapted to move an endoscope along a lumen by pulling the distal end of the endoscope. According to one mode of this aspect, by pulling the distal end of the endoscope, the endoscopic delivery assembly substantially limits the stretching of the lumenal wall during delivery. According to another aspect, an endoscope delivery assembly and method is adapted to deliver an endoscope along a luminal wall with substantially limited risk of organ wall perforation. According to another aspect, an endoscope delivery assembly and method is provided that is adapted to substantially decrease procedure related pain. According to one mode of this aspect, the substantially decreased procedure-related pain is achieved by substantially reducing the extent to which the lumen wall is stretched during endoscope delivery.

Another aspect of the invention provides a colonoscopy system and method that incorporates a colonoscope delivery assembly. According to one mode of this aspect, the colonoscope delivery assembly is adapted to allow enhanced patient comfort during colonoscopy with substantially limited sedation.

Another aspect of the invention provides a colonoscopy system and method that is adapted to allow colonoscopy to be performed without substantial sedation. According to one mode of this aspect, such system and method is adapted to be used at lower cost facilities, such as for example a physician's office, than is generally accepted according to other conventional colonoscopy systems and methods.

Another aspect of the invention provides an endoscope delivery assembly and method that is adapted to move an endoscope along a body lumen without substantially changing the length of the endoscope. According to one mode of this aspect, the endoscope delivery system and method is adapted to move a commercially available endoscope in this manner. According to another mode of this aspect, as the length of the endoscope remains substantially fixed, one or more commercially available endosurgical devices, such as in certain beneficial embodiments polypectomy snares and biopsy forceps, are provided and/or used in conjunction with the system and method.

Another aspect of the invention provides an endoscope delivery assembly that is adapted to provide for the further combination and use of endosurgical devices and methods, including for example both diagnostic and therapeutic devices and related procedures. Another aspect of the invention provides an endoscope delivery assembly that is adapted to decrease procedure-related risk by decreasing the incidence of perforation during endoscopy. According to one mode, perforation is substantially reduced according to the assembly by pulling the endoscope at its distal end and by using an annular invaginating balloon as a tracking mechanism.

Another aspect of the invention provides an endoscope delivery assembly with an annular invaginating balloon that, in a radially collapsed configuration, has a first diameter that is sufficiently small to provide for introduction into a body lumen. After insertion, the annular invaginating balloon is inflated to a radially expanded configuration that is adapted to contact the luminal wall.

According to another aspect of the invention, an endoscope delivery assembly and method provides an invaginating balloon that has a removable inflation device. According to one mode, the removable inflation device comprises a cannula. According to another mode of this aspect, the balloon surface is sufficiently smooth so as to substantially limit risk of trauma to the lumen wall.

According to another aspect of the invention, an endoscope delivery assembly and method provides an annular invaginating balloon that circumscribes a longitudinal axis and has a cross-sectional profile substantially in the shape of a toroid. According to one highly beneficial mode of this aspect, the toroidal shape of the annular invaginating balloon has a length along the longitudinal axis that is larger than the cross-sectional diameter through a portion of the wall of the balloon in a radial axis transverse to the longitudinal axis, e.g., a length dimension that is longer than a simple toroid shaped balloon, thus forming an elongate tube with a lumen extending therethrough.

According to another aspect of the invention, an endoscope delivery assembly and method provides an annular invaginating balloon that rotates about its long axis while making contact with the respective lumen wall. In one highly beneficial mode of this aspect, the rotating annular invaginating balloon is adapted to provide for rolling traction of the assembly, and related assemblies coupled therewith, along the lumen wall. According to another mode, the annular invaginating balloon functions like a wheel in contact with the lumen wall. The annular invaginating balloon is a dynamic part of the endoscope delivery assembly and provides rolling traction along the wall, resulting in movement of the endoscope delivery assembly and respectively coupled components and assemblies, e.g., such as an endoscope shaft or endoscope delivery cannula coupled thereto, along the lumen.

Another aspect of the invention provides an endoscope delivery assembly that is under substantial direct control of the endoscopist. Additional aspects of the invention include various respective methods of operating the assemblies noted herein, which methods generally augment or replace various aspects of the endoscopic procedures and techniques previously available.

Another aspect of the invention provides an endoscope delivery assembly that incorporates a relatively simple machine with relatively few working parts. Another aspect of the invention provides an endoscope delivery assembly that is sufficiently simple so as to allow for a relatively low cost of production as compared to other endoscope delivery assemblies intended to augment traversal of various tortuous lumens, such as for example the colon.

Another aspect of the invention provides an endoscope delivery assembly that can be manufactured at sufficiently low cost so as to allow for a disposable product. According to one mode of this aspect, providing the endoscope delivery assembly as a disposable product substantially reduces the risk of infectious disease transmission, such as for example from one patient to another as may occur with higher cost equipment that is thus re-used over multiple patients.

Another aspect of the invention provides an endoscope delivery assembly that includes an integral sheath and at least one attachment bracket insure ease of attachment to an endoscope and safety of operation. Another aspect of the invention is an endoscope propulsion device assembly with a toroidal wall, a drive assembly, and an endoscope coupler assembly as follows. The toroidal wall has an exterior surface and an interior surface that circumscribes an interior passageway extending along a longitudinal axis, and with a length between a proximal end and a distal end relative to the longitudinal axis. The toroidal wall is adjustable from a radially collapsed condition to a radially extended condition, respectively, transverse to the longitudinal axis. The drive assembly is adapted to couple to the toroidal wall and to impart toroidal rotation onto the toroidal wall in the radially extended condition such that the interior surface translates in a first longitudinal direction and the exterior surface translates in a second opposite longitudinal direction along the longitudinal axis. The endoscope coupler assembly is adapted to couple the toroidal wall to an endoscope extending along the interior passageway such that the toroidal wall and endoscope are adapted to be propelled together in the first direction along a body lumen during toroidal rotation of the toroidal wall when the exterior surface is engaged to a wall of the body lumen with translating force against the wall. According to one mode of this aspect, the toroidal wall is provided in the form of a toroidal balloon. In another embodiment, this toroidal balloon has an annular invaginated balloon wall and is inflatable from the radially collapsed condition to the radially extended condition with a pressurized fluid. In another mode, the toroidal balloon includes a protrusion extending from the balloon wall along the interior surface and into the interior passageway. The drive assembly is provided with an elongate screw extending along the longitudinal axis within the interior passageway and with a helical groove extending helically around the longitudinal axis. This helical groove is adapted to receive the protrusion within the interior passageway such that rotation of the elongate screw advances the protrusion longitudinally in the first direction along the longitudinal axis. The helical groove is thus adapted to move the interior surface in the first direction along the longitudinal axis to impart toroidal rotation to the toroidal balloon along the longitudinal axis.

According to one further embodiment of this mode, the protrusion extends from the interior surface with a relatively narrow neck and terminates interiorly within the interior passageway with an enlarged head relative to the neck. According to another embodiment, a plurality of such protrusions are provided in a patterned group that are each spaced along a longitudinal pattern that circumscribes one lobe of the toroidal balloon along the longitudinal axis. Each protrusion of the group along the interior surface is engaged to a respective turn of the helical groove and translates longitudinally in the first direction along the rotating screw. Each protrusion of the group along the inner surface is released from the helical groove when it is translated in the first direction to a first end of the screw; whereas each protrusion of the group along the exterior surface translates in the second opposite direction and is adapted to rotate inwardly to the inner surface and to be engaged within the helical groove of the screw at a second end thereof. Accordingly, continuous rotation of the screw continuously releases and engages respective protrusions of the patterned group at the first and second ends of the screw, respectively, to continuously drive toroidal rotation of the toroidal balloon. According to one further feature that may also be provided according to this embodiment, a plurality of such groups of protrusions is provided in respectively patterned arrays. Each of the groups of protrusions is located at a unique respective position around a circumference of the toroidal balloon transverse to the longitudinal axis.

According to another further feature, four of such groups of protrusions are provided. In still a further feature, these may be spaced at 90 degree intervals around the circumference transverse to the longitudinal axis. In still another feature, a cowling with a substantially tubular body is located between the screw and the interior surface of the toroidal balloon and includes a longitudinal groove extending along the longitudinal axis between first and second ends of the screw. The protrusions are adapted to engage the helical groove of the screw through the longitudinal groove of the cowling. In another feature related to multiple groups of protrusions, a cowling with a substantially tubular body is located between the screw and the interior surface of the toroidal balloon and with a plurality of longitudinal grooves extending along the longitudinal axis between first and second ends of the screw. The protrusions of each group are adapted to engage the helical groove of the screw through a respective one of the plurality of longitudinal grooves of the cowling.

According to another embodiment related to inflatable toroidal balloon modes of this aspect, an expansion actuator is also provided that is adapted to couple to the toroidal wall and expand the toroidal wall from the radially collapsed condition to the radially extended condition. According to another mode, a motor is also provided that is adapted to couple to the drive assembly and to actuate the drive assembly coupled to the toroidal wall to impart toroidal rotation to the toroidal wall. According to yet another mode, an endoscope is also provided in the system. According to one embodiment of this mode, the endoscope and the toroidal wall are permanently secured in fixed position relative to each other via the endoscope coupler assembly. In another embodiment, the endoscope and toroidal wall are adapted to be releasably coupled to each other via the endoscope coupler assembly. According to another mode, the endoscope coupler assembly includes a base with a tubular member with an inner lumen extending along a length between first and second ends. The coupler assembly also includes first and second radial protrusion stops extending radially outwardly from the tubular member transverse to the longitudinal axis at each of the first and second ends, respectively. The base is adapted to be coupled to an endoscope extending along the inner lumen. The toroidal wall is adapted to be positioned at a location along the base with the tubular member located within the interior passageway and such that in the radially extended condition the toroidal wall has an inner diameter at the interior surface that is less than an outer diameter of the base at the first and second radial protrusion stops. The toroidal wall is adapted to undergo toroidal rotation at the position without substantially moving longitudinally along the base due to mechanical interference between the toroidal wall and the first and second radial protrusion stops.

According to another embodiment of the inflatable toroidal balloon mode, the drive assembly includes a belt that circumscribes one lobe of the toroidal balloon wall along the longitudinal axis and at a position around the circumference transverse to the longitudinal axis. The toroidal balloon wall includes a circumferential groove along the longitudinal axis and corresponding with the position. The belt is adapted to engage the circumferential groove along the exterior surface of the toroidal balloon wall at the position. The belt is also adapted to engage the drive assembly located within the interior passageway. The drive assembly is adapted to rotate the belt around the toroidal balloon and so as to impart translational motion to the exterior surface in the second direction to thereby provide toroidal rotation of the balloon.

In one further feature of this embodiment, the groove has a shaped interior surface with a plurality of spaced pairs of opposite protrusions into the groove to provide an alternating pattern of expanded and narrowed waste regions along the groove. The belt has a shaped outer surface with a plurality of enlargements separated by relatively narrowed waste regions. The belt and groove are adapted to couple along the exterior surface with the narrowed waste regions of the belt fitting into the narrowed waste regions of the groove. The belt is adapted to be released from the groove at first and second ends of the exterior surface along the balloon. According to another mode, the toroidal wall comprises an elongated toroidal wall such that the length is substantially greater than a profile diameter between the interior and exterior surfaces of the toroidal wall in the radially extended condition.

Another aspect of the invention is a method for propelling an endoscope. This method includes coupling a toroidal wall to an endoscope at a location along a distal end portion of the endoscope, coupling a drive assembly to the toroidal wall at the location, and adjusting the toroidal wall from a radially collapsed condition to a radially extended condition, respectively, transverse to the longitudinal axis at the location. The drive assembly is actuated to impart toroidal rotation onto the toroidal wall in the radially extended condition at the location such that the interior surface translates in a first longitudinal direction and the exterior surface translates in a second opposite longitudinal direction along the longitudinal axis. In addition, the toroidal wall is substantially maintained at the location along the endoscope while imparting the toroidal rotation to the toroidal wall. According to one mode of this aspect, the endoscope and respectively coupled toroidal wall and drive assembly are inserted into a body lumen of a patient. A lumen wall of the body lumen is engaged with the exterior surface of the toroidal wall in the radially extended condition. The toroidal wall and endoscope are propelled together in the first longitudinal direction along the body lumen by imparting the toroidal rotation to the toroidal wall and thereby translating the exterior surface with force in the second opposite direction against the respectively engaged body lumen wall.

Another aspect of the invention is a method for performing endoscopy within a body lumen in a patient as follows. An endoscope assembly, preferably sterile or having been sterilized, is inserted within the body lumen. A substantial circumference of a body lumen wall of the body lumen surrounding the endoscope is engaged with a propulsion assembly coupled to the endoscope. An axial force against the body lumen wall and around the substantial circumference is provided with the propulsion assembly. Accordingly, the endoscope is propelled along the body lumen at least in part using the axial force against the body lumen wall from the propulsion assembly.

According to further aspects of the invention, the various other aspects herein described for an endoscope delivery assembly, its construction, and the various related aspects and modes of method of operation, are suitably modified and applied to non-medical uses. In certain further modes of this aspect, such assemblies and methods are incorporated into devices and methods for visual inspection and manipulation of other tubular structures. It is also to be appreciated that each of the foregoing aspects, modes, embodiments, variations, features, or variants on such features is to be considered independently useful without necessarily requiring combination with the others unless expressly stated so. Notwithstanding the foregoing, it is also further appreciated that the various combinations and sub-combinations between them, as would be apparent to one of skill in the art, are further considered independently useful and within the intended scope hereof.

Turning now to the figures, which depict certain exemplary embodiments of the invention, for illustrative purposes, embodiments of the present invention are depicted in the apparatus generally shown in FIG. 1 through FIG. 9. It will be appreciated that the apparatus may vary as to configuration and as to details of the parts, and that the method may vary as to the specific steps and sequence, without departing from the basic concepts as disclosed herein.

As used herein, an “annular invaginating balloon” is generally a balloon which has a cross-sectional profile that is donut shaped like a toroid. However, in contrast to a toroid, this variation has a length that is greater than its diameter. The balloon generally functions as an active, dynamic component of an endoscope delivery assembly, and in many instances an endoscopic propulsion device, and provides rolling traction like a wheel or tire. In embodiments, the membranous element or annular balloon can be fixed (i.e., not invaginating) but can cooperate with other structure to provide rolling traction, such as one or more belts (e.g., belts with tread) that circumscribe the fixed balloon/membranous element. In such embodiments, instead of the annular balloon invaginating within itself to provide the rolling motion, such belts will be caused to rotate around the balloon by way of interaction with a functionaldrive, for example a worm drive or other drive mechanism, including a friction drive, magnetic drive, or direct gear drive, to provide the rolling traction. In further embodiments, the annular balloon/membranous element can be capable of invaginating (i.e., not fixed) as well as capable of cooperating with one or more of such belts to provide, in addition to the belts, another active component of the system. In such embodiments, the annular invaginating balloon would move in the same direction as the belts to provide additional rolling traction. Where it is not specified, a membrane of the invention may be any toroidal shape, including, but not limited to an annular invaginating balloon.

As used herein, an “endoscope” is generally intended to mean an optical or video device for examining the lumen (internal opening) of an organ. A “fluid” according to the invention is a material that is capable of flowing, not solid of static shape and form; and may be liquid or gaseous (Funk and Wagnalle, “Standard College Dictionary” Harcourt, Brace & World cw1968). Further, the term “gear” is intended to mean a device adapted to interact in a mechanical assembly of interacting parts that serves to transmit motion or to change the rate or direction of motion (Funk and Wagnalle, “Standard College Dictionary” Harcourt, Brace & World cw1968). Term “helical gear” is intended to mean a gear having teeth arranged in the configuration of a helix. (“Machinery's Handbook” 25 ed., Industrial Press Inc. New York, 1996.) The term “motor” is intended to mean something that imparts or produces motion (Funk and Wagnalle, “Standard College Dictionary” Harcourt, Brace & World cw1968). The term “pin coupling” is intended to mean a form of slip joint coupling to a shaft of a motor. The term “pinion gear” is intended to mean a toothed wheel driving or driven by a larger cogwheel (Funk and Wagnalle, “Standard College Dictionary” Harcourt, Brace & World cw1968), while the term “rolling traction” or “rotary traction” is intended to mean the act of drawing, as by motive power over a surface using rolling or rotational movement, respectively, such as a wheel or tire. Finally, the term “toroid” is intended to mean a surface generated by the rotation of any closed plane curve about and axis lying in its plane but external to it (e.g. donut shaped) (Funk and Wagnalle, “Standard College Dictionary” Harcourt, Brace & World cw1968).

FIG. 1 shows a perspective view of one embodiment of a device 2100 according to the invention, in particular, an endoscope and cooperating propulsion system (drive unit). As shown in FIG. 1, endoscope 2120 is housed within supports 2151, which also support a portion of the drive unit. In this embodiment, the drive unit includes a functional drive, for example a worm drive (not depicted) housed within cowling (outer cylinder) 2152. Other suitable functional drives for this and every embodiment described herein include friction drives, magnetic drives, and direct gear drives to name a few. Cowling 2152 of the drive unit provides protection to the components of the drive unit as well as provides support for belts 2111 during engagement with the functional drive, e.g., a worm drive. In this embodiment, belts 2111 comprise tread 2112 for engagement with a functional drive and engagement with a surface, such as the internal surface of a cavity, e.g., the gastrointestinal tract of a human. Suitable tread for this and every embodiment described herein provides a traction force sufficient provide mobility with little or no detrimental impact on the lumen. During operation of the device, a drive shaft 2141, in combination with gears, rotates a functional drive, which rotates engaged belts 2111. Drive shaft 2141 can be a flexible draft shaft connected to a motor that is maintained outside of the patient's body. Tread 2112 of engaged belts 2111 provides for longitudinal movement of device 2100 by engaging the internal surface of a cavity. More specifically, as engaged belts 2111 are caused to rotate by the functional drive, tread 2112 provides traction for longitudinal movement of device 2100 against the opposing internal surfaces of a cavity. In this embodiment, tread 2112 comprises castellated projections, which communicate and are engaged with the functional drive to cause rotation of the belts. Membranous element 2130 in this embodiment is an inflatable balloon that is circumscribed by belts 2111. Balloon 2130 can be inflated or deflated, as desired, to increase or decrease pressure of tread 2112 against opposing internal surfaces of a cavity. Inflating or deflating the balloon may be desired, for example, in situations where the cavity being traversed by device 2100 increases or decreases in size.

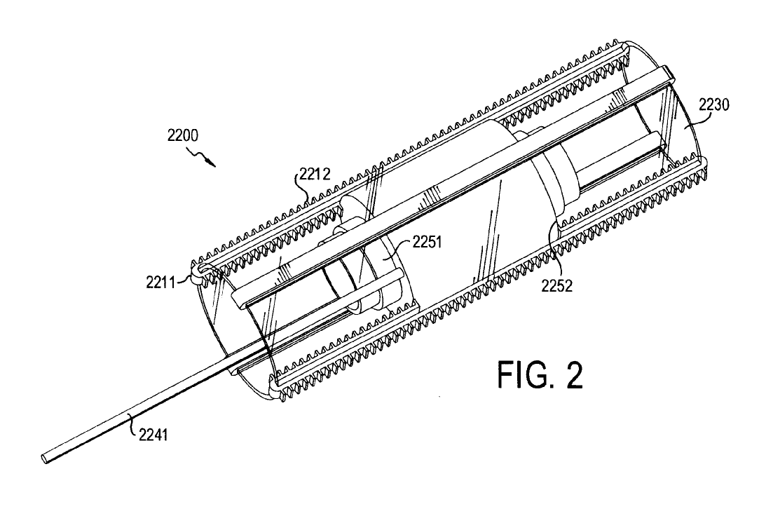

FIG. 2 shows a perspective view of one embodiment of a propulsion system according to the invention, in particular, the device of FIG. 1 without the endoscope. As described previously, the propulsion system can be adapted for numerous applications, not limited to the art of medical devices. Propulsion system (drive unit) 2200, as shown in FIG. 2, comprises drive shaft 2241 for converting electrical power to rotational energy. Drive shaft 2241, in combination with gears, communicates with a functional drive (not shown) housed within cowling 2252 and supported by supports 2251. The functional drive communicates with belts 2211, and in this embodiment communicates particularly with tread 2212, to rotate belts 2211. Rotating belts 2211 communicate with the internal surface of a cavity to provide longitudinal movement to device 2200 within the cavity. The castellated projections of tread 2212, as shown in this embodiment, provide for traction with the internal surface of the cavity, as well as for engagement means for communication with the functional drive. Friction between tread 2212 and the internal surface of a cavity can be increased or decreased with balloon 2230, if desired. For example, when balloon 2230 is inflated, pressure is imposed on belts 2211 against opposing walls of the cavity to produce additional traction. Balloon 2230 can also be fixed, for example, secured to cowling 2252 and/or supports 2251, so that belts 2211 rotate around the external surface of balloon 2230, while balloon 2230 remains stationary. Balloon 2230 can also be an annular invaginating balloon, e.g., unsecured to any support or secured to belts 2211. In such embodiments, an annular invaginating balloon 2230 can provide additional traction and/or reduce drag resulting from contact of balloon 2230 with the internal surface of a cavity, depending on the characteristics of the surface of the balloon and whether that surface provides more or less friction than with the belts alone.

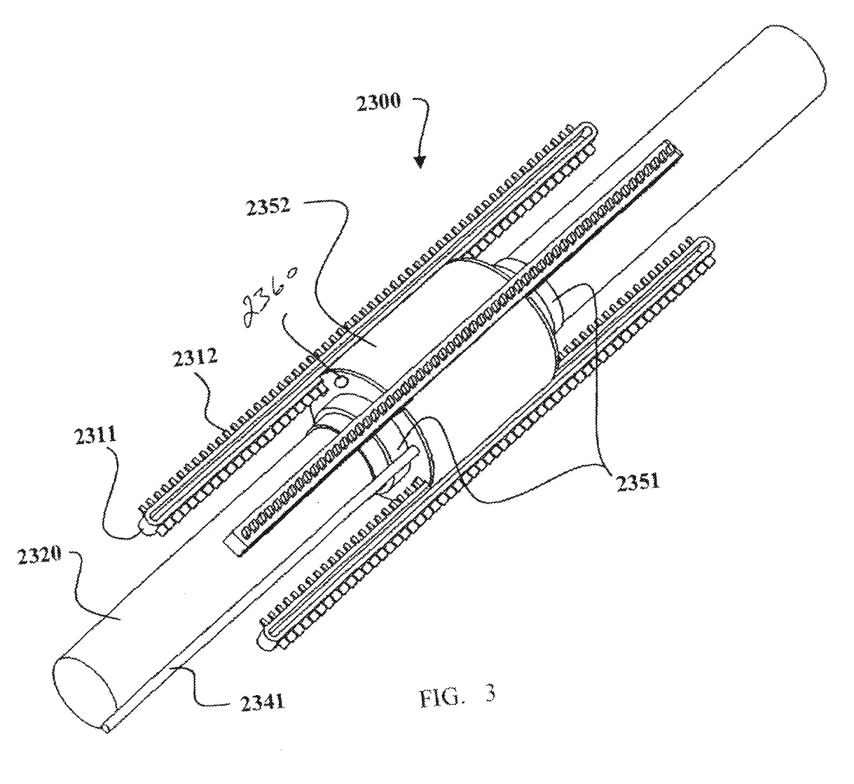

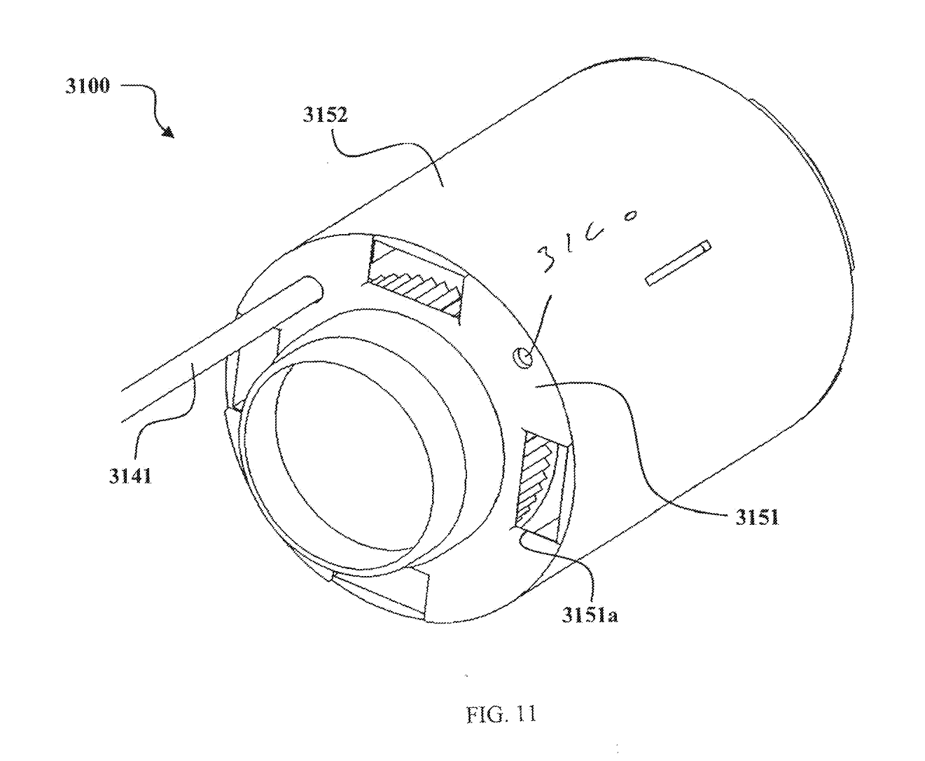

FIG. 3 shows a perspective view of device 2300, which is one embodiment of an endoscope and cooperating propulsion system according to the invention, in particular, the device according to FIG. 1 with some components removed to view more internal components of the device. As shown, endoscope 2320 is attached to the drive unit at supports 2351. Supports 2351 are substantially the same; however, proximal support 2351 provides a hole for drive shaft 2341 to pass through for communication with the functional drive. Distal support 2351 need not comprise such a hole. The terms proximal and distal as used with respect to supports 2351 and similar supports in other figures in this application refer to orientation of the device with respect to the surgeon operating the device in a typical situation. Cowling 2352 protects internal components of the drive unit and holds belts 2311 in place for communication with the functional drive (not shown). Tread 2312, when rotated by the functional drive, provides longitudinal movement to device 2300 within a cavity. In this embodiment, tread 2312 comprises castellated projections for such communication. Of particular interest in FIG. 3, is that belts 2311 comprise a flexible material, for example, an inert rubber, so that belts 2311 can adapt and/or conform to the shape of a particular cavity by being traversed by the device. Proximal support also has an opening 2360 for an air insufflations tube (not shown). The air insufflation tube runs through the proximal support and attaches to the cowling which has a communication to the inner surface of the balloon to allow inflation and deflation of the balloon. This also allows monitoring of balloon pressure. As shown in FIG. 11, which will be described in detail below, this insufflation tube connection is located on the cowling communicating to the outer surface of the cowling; this in turn communicates with the balloon. Two holes are present in the proximal support (one for the drive shaft and one for the air insufflation tube) with the air insufflation tube running through one of these holes and connecting to the cowling.

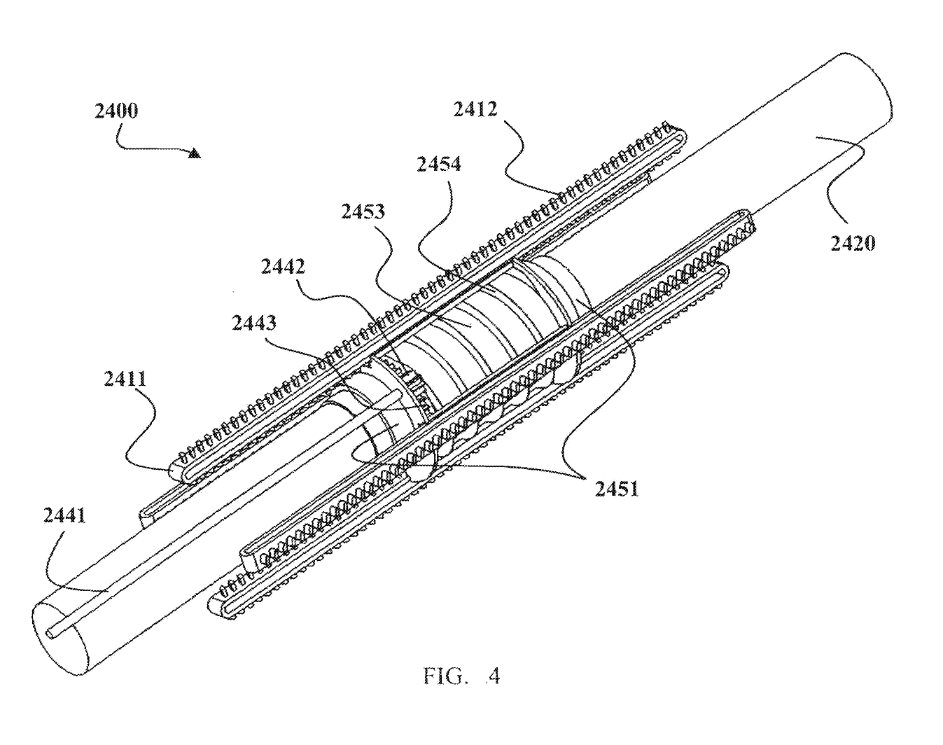

FIG. 4 shows a perspective view of one embodiment of an endoscope and cooperating propulsion system according to the invention, in particular, the device according to FIG. 1 and FIG. 3 with components removed to view other components of the device. Of particular interest in FIG. 4, the cowling of the drive unit of device 2400 has been removed to show the internal components of the drive unit. As shown, supports 2451 provide support for other components of the drive unit, including a functional drive, e.g., worm drive 2453, cowling (not shown), and drive shaft 2441. In this embodiment, drive shaft 2441 is secured to or incorporates a first gear 2442, which rotates with the drive shaft. First gear 2442 communicates with second gear 2443, which is secured to or part of functional drive 2453. Structure 2454 communicates with belts 2411, in particular tread 2412 (e.g., castellated projections), to rotate belts 2411. More particularly, a motor converts electrical energy to rotational energy of drive shaft 2441, which by way of first gear 2442 and second gear 2443 rotates functional gear 2453, which comprises complementary structure 2454 to engage belts 2411 at tread 2412. The number of gears, type of functional drive (e.g., worm drive), and configuration of the cooperating parts of the drive unit are not critical. Thus, any combination or number of drive components, including gears, can be used, so long as the drive unit comprises means for converting electrical energy to rotational energy and ultimately to longitudinal movement of the device within or through a cavity. In this embodiment, tread 2412 consequently engages the interior surface of a cavity to move endoscope 2420 in a substantially longitudinal direction through and within a cavity, e.g., a gastrointestinal tract. A separate view of the gearing is shown in FIG. 15.

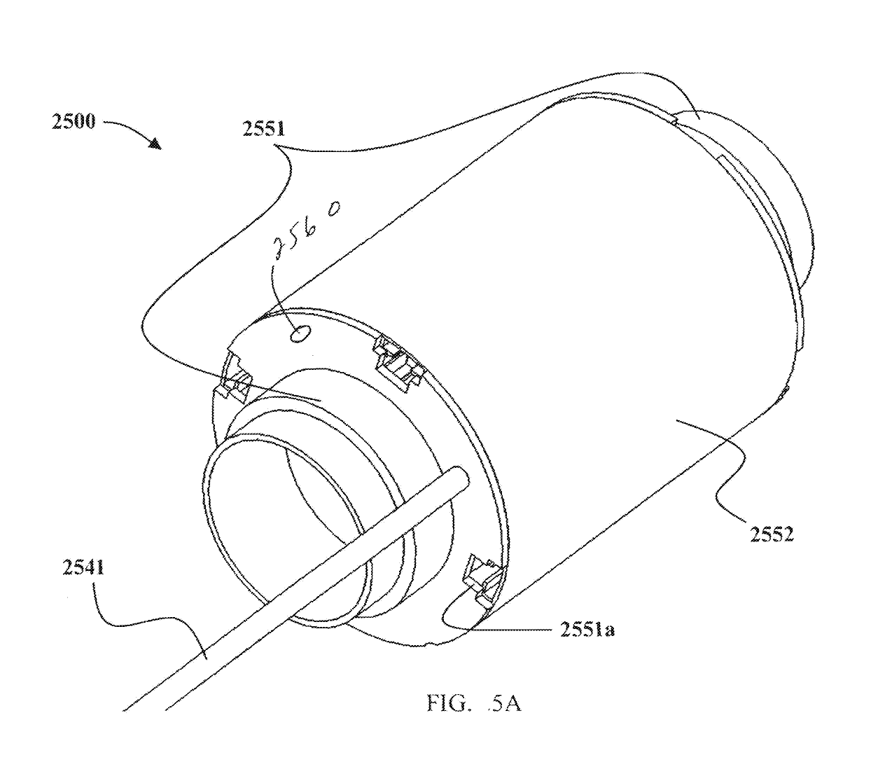

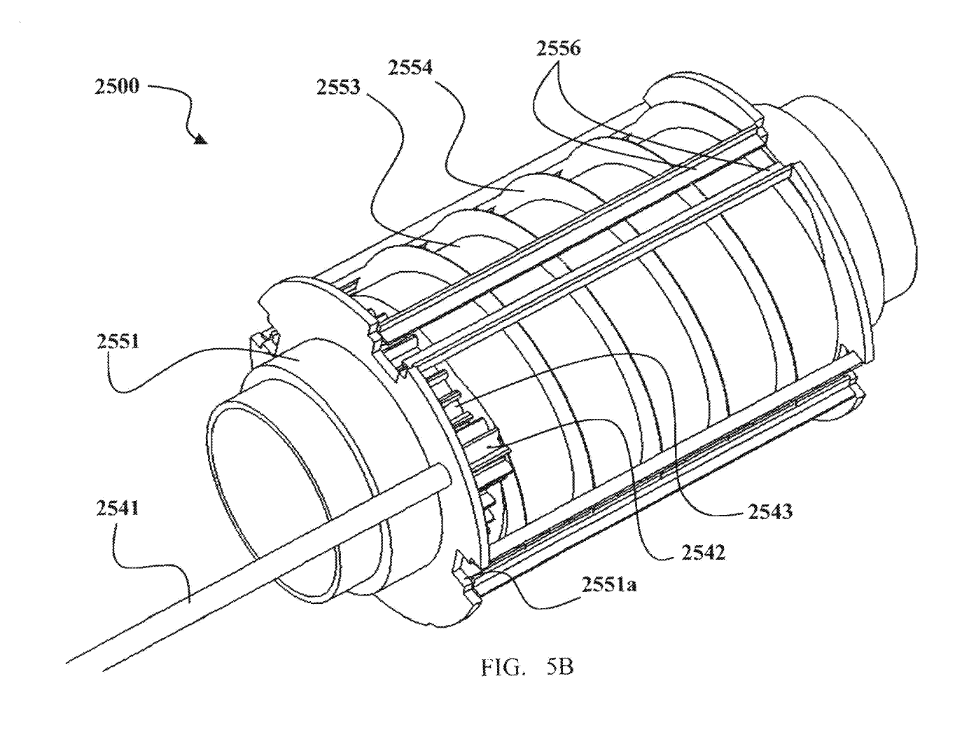

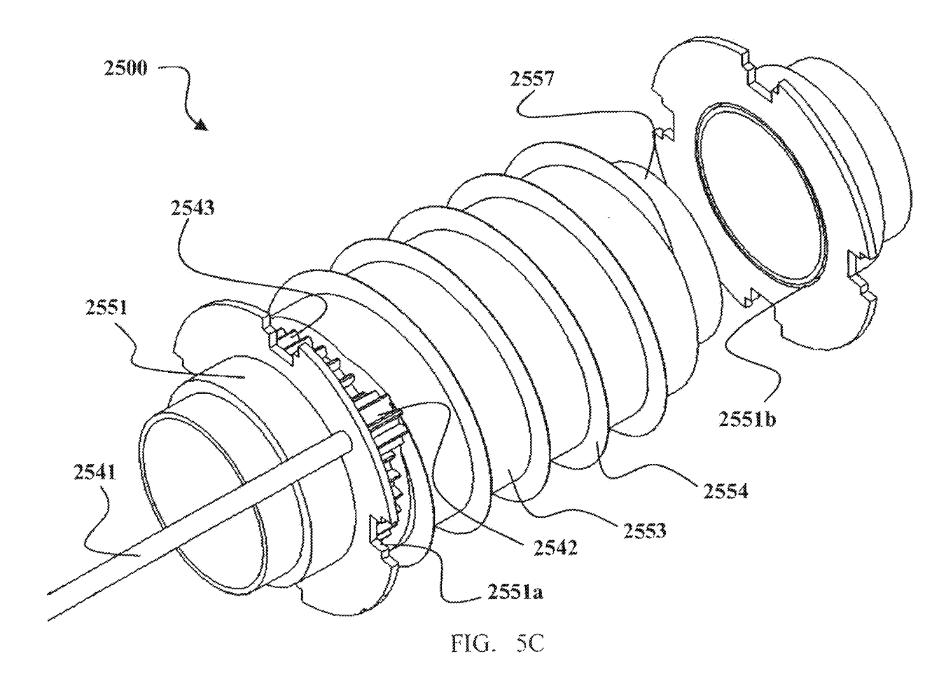

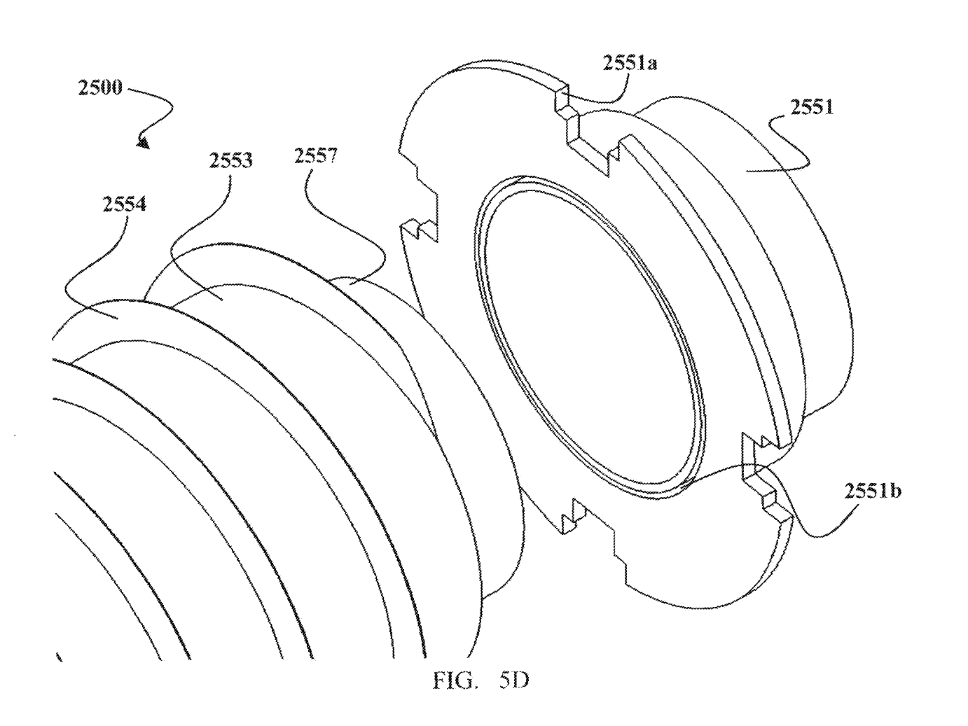

FIG. 5A shows a perspective view of the housing for a portion of the drive unit. As shown in FIG. 5A, drive unit 2500 comprises a cowling (outer cylinder) 2552 and supports 2551 for housing and supporting components of the drive unit. Proximal support 2551 comprises means for allowing drive shaft 2541 to pass through (e.g., a hole). Proximal and distal supports 2551 support the internal components of the drive unit, including the functional drive (not shown), and notches 2551a in supports 2551 provide support for the belts that interact with the functional drive. Drive shaft 2541 passes through proximal support 2551 to interact with the functional drive, typically, by way of cooperating gears. As explained above, an opening 2560 for an air insufflation tube is provided.

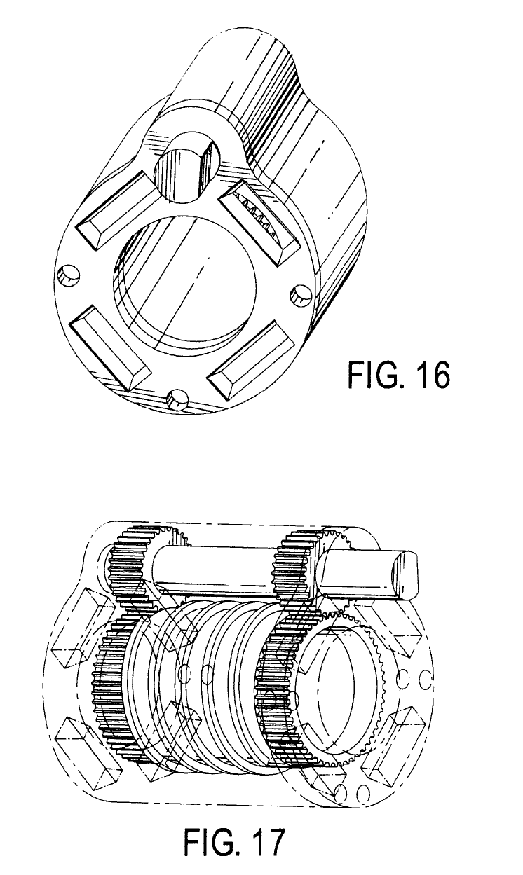

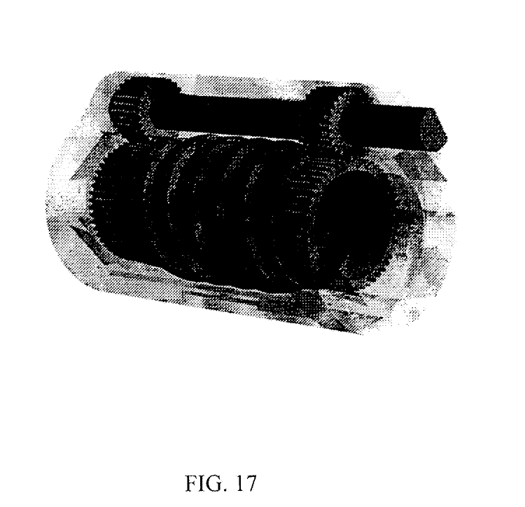

FIG. 5B shows a perspective view of a portion of the drive unit, including the drive shaft, drive gears, functional drive, and housing. More particularly, the cowling of drive unit 2500 has been removed to show the internal components of the drive unit. As shown in FIG. 5B, the drive unit includes a drive shaft 2541, which passes through proximal support 2551 and which comprises or is attached to a first gear 2542. First gear 2542 communicates with second gear 2543, which is secured to or is comprised in functional gear 2553. Functional gear 2553 comprises corresponding structure 2554 for engaging flexible belts (not shown), for example, at tread (belts with tread not shown). Flexible belts are supported by belt guides 2556, which provide assistance in keeping the belts engaged with functional gear 2553 during operation of the unit. As shown, belt guides 2556 are supported by notches in supports 2551 at notch 2551a. In this embodiment, the belts are supported (or would be if shown) by (e.g., rest on) belt guides 2556 and would be held in place by the cowling, which provides minimal back pressure to the opposing side of the belts. In this embodiment, the belts would be sufficiently supported (but not fixed in place) by belt guides 2556 and the cowling to provide for interaction with the functional drive, while allowing for the belts to rotate. Other views are shown in FIGS. 16 and 17.

FIG. 5C shows a perspective view of a portion of the drive unit, including the drive shaft and gears, the functional drive, and housing. More particularly, drive unit 2500 includes a drive shaft 2541, which comprises or is secured to a first gear 2542 for engaging a second gear 2543. Drive shaft 2541 passes through proximal support 2551 to engage second gear 2543. Second gear 2543 is secured to or is comprised in functional drive 2553. Functional drive 2553 comprises corresponding or complementary structure 2554 for engaging flexible belts, for example, flexible belts with tread. Functional drive 2553 rests and rotates around a concentric shaft, otherwise referred to as inner cylinder 2557. Inner cylinder 2557 is secured and supported by supports 2551 at grooves 2551b. Notches 2551a in supports 2551 provide support for the flexible belts. When engaged by drive shaft 2541, by way of gears 2542 and 2543, functional drive 2553 engages flexible belts, which produce substantially longitudinal movement of the device through and within a cavity. As used in this application, the term longitudinal or substantially longitudinal is used to refer to the overall movement of the device through or within a cavity, even though it is recognized that the device may also rotate within the cavity while moving longitudinally during operation. Thus, although the path of the device through and within a cavity may not be exactly longitudinal, what is meant by longitudinal or substantially longitudinal in the context of this invention is that the device is capable of achieving some overall longitudinal distance within the cavity.

FIG. 5D shows a perspective view of a portion of drive unit 2500, in particular, the functional drive and cooperating supports. This view shows functional drive 2553 comprising corresponding structure 2554, wherein functional drive 2553 is supported by inner cylinder 2557. Inner cylinder 2557 is supported by supports 2551 at both the proximal and distal ends of the device at groove 2551b. Shown in this embodiment is distal support 2551. Also shown are notches 2551a of supports 2551, which provide support for the belt guides (not shown) and ultimately the flexible belts which would rest on the belt guides.

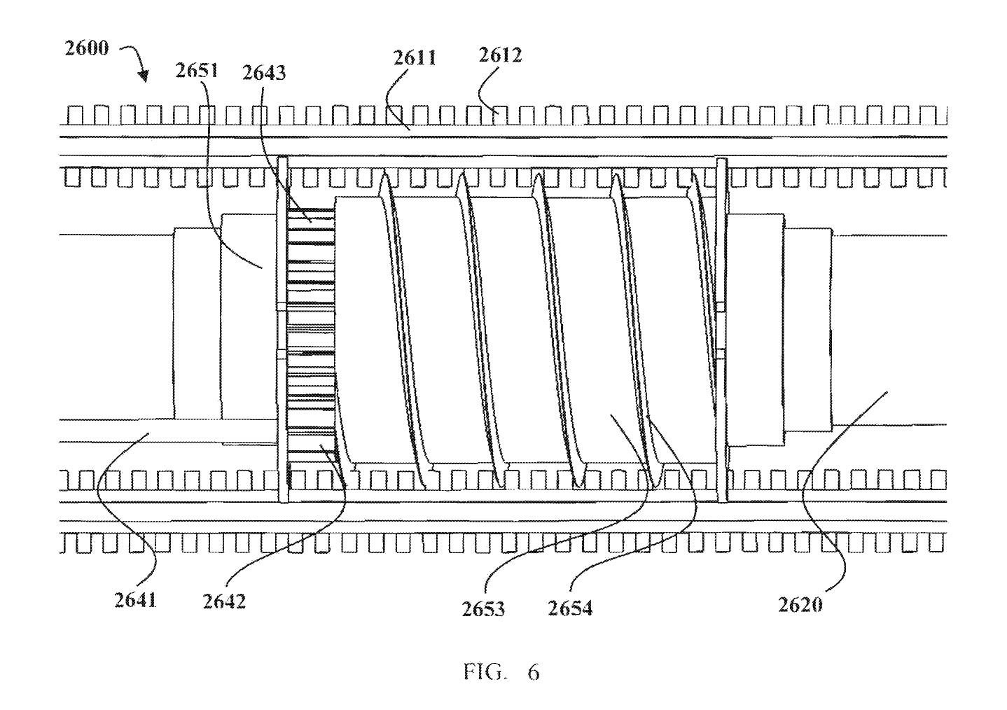

FIG. 6 shows a side view of a portion of the drive unit, including the drive shaft and gears, the functional drive, and cooperating flexible belts with tread. More particularly, FIG. 6 shows device 2600, which comprises a drive unit and endoscope 2620, which are connected by supports 2651. The drive unit is powered electrically by a motor, which drives drive shaft 2641. Drive shaft 2641 comprises or is secured to a first gear 2642, which engages a second gear 2643. Second gear 2643 is comprised in or is secured to functional drive 2653. Functional drive 2653 comprises corresponding complementary structure 2654 for engaging flexible belts 2611. Flexible belts 2611 comprise tread 2612 for engagement with functional drive 2653 and for engagement with the internal surface of a cavity, such as a gastrointestinal tract. As shown in the embodiment, tread 2612 can comprise castellated projections for such engagement.

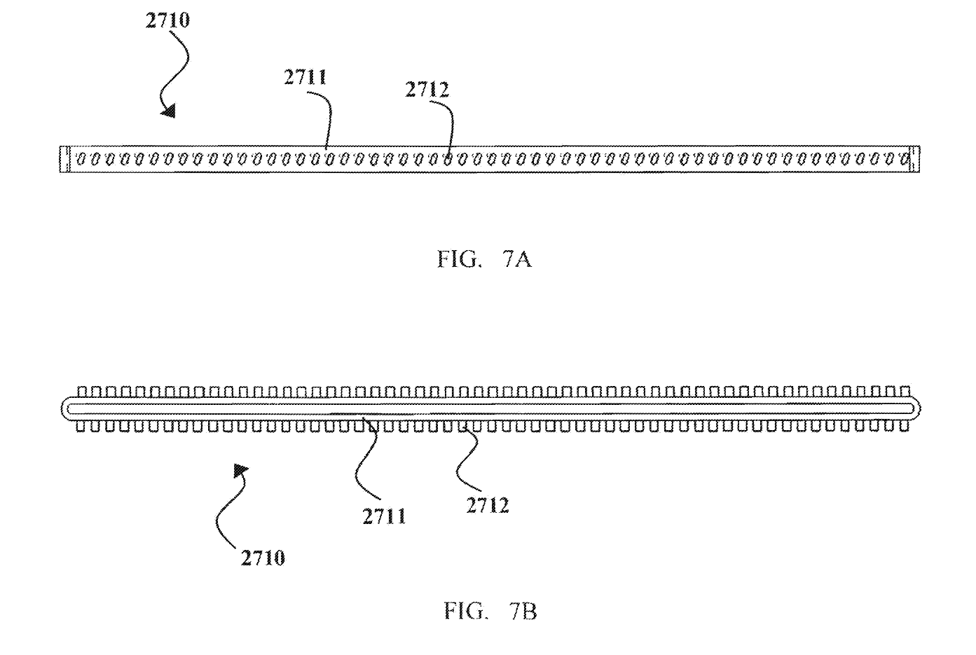

FIGS. 7A and 7B show, respectively, a top and side view of a flexible belt. As shown, flexible belt 2710 comprises belt 2711, which is made of a flexible material. One of skill in the art would understand which materials are best suited for a particular application, however, any flexible material, especially materials inert to the human body, can be used, including any such material described in this application, especially with respect to the membranous element. Belts 2711 comprise tread 2712, which need not conform to any particular shape or arrangement so long as tread 2712 functions to engage with the functional drive and engage with the internal surface of a cavity to provide propulsive movement of the device during operation. In this embodiment, tread 2712 comprises castellated projections for such engagement. Further, although four belts are typically used in the embodiments described in this application, any number of belts can be used depending on a particular application. More particularly, devices of the present invention can comprise, for example, 1, 2, 3, 4, 5, 6, 7, 8, 9, or 10 belts, but typically no more than 20 belts. One of skill in the art will recognize the appropriate number of belts called for in a particular application. Further, as discussed previously within this application and depending on the particular characteristics of the materials chosen and/or the particular application, the belts need not comprise tread.

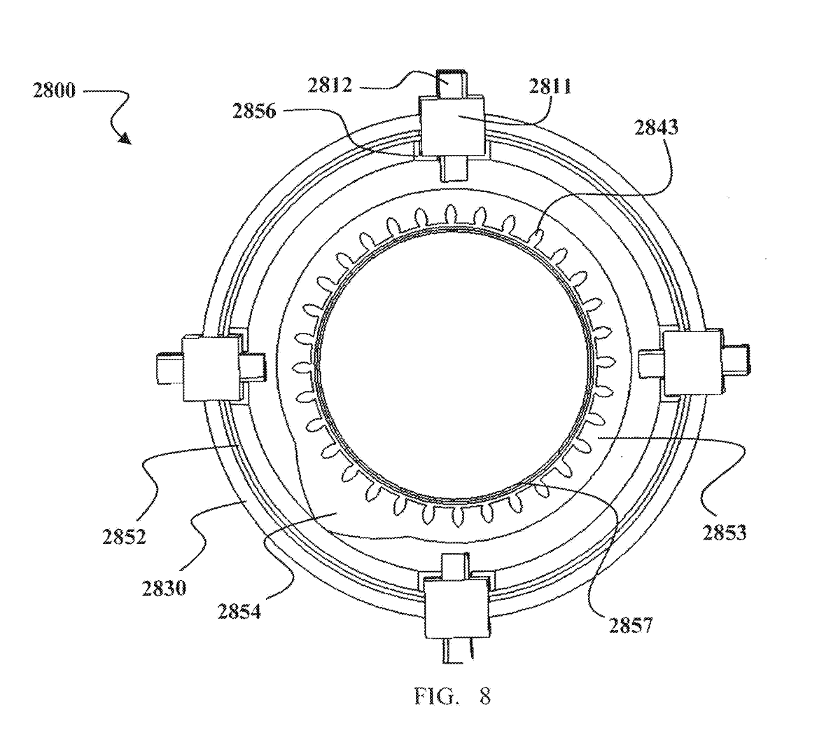

FIG. 8 shows a cross-sectional view of a portion of the drive unit, including the functional drive and gears, cooperating belts, and balloon. Drive unit 2800, as shown, comprises inner cylinder 2857, which supports gear 2843 and functional drive 2853 having corresponding structure 2854. Functional drive 2843 engages belts 2811 at tread 2812 (here, castellated projections). Belts 2811 are supported by belt guides 2856 and on the opposing side of the belt by cowling 2852. Balloon 2830 rests on or is secured to cowling 2852 and provides flexible belts 2811 with opposing pressure when engaging the inner surface of a cavity.

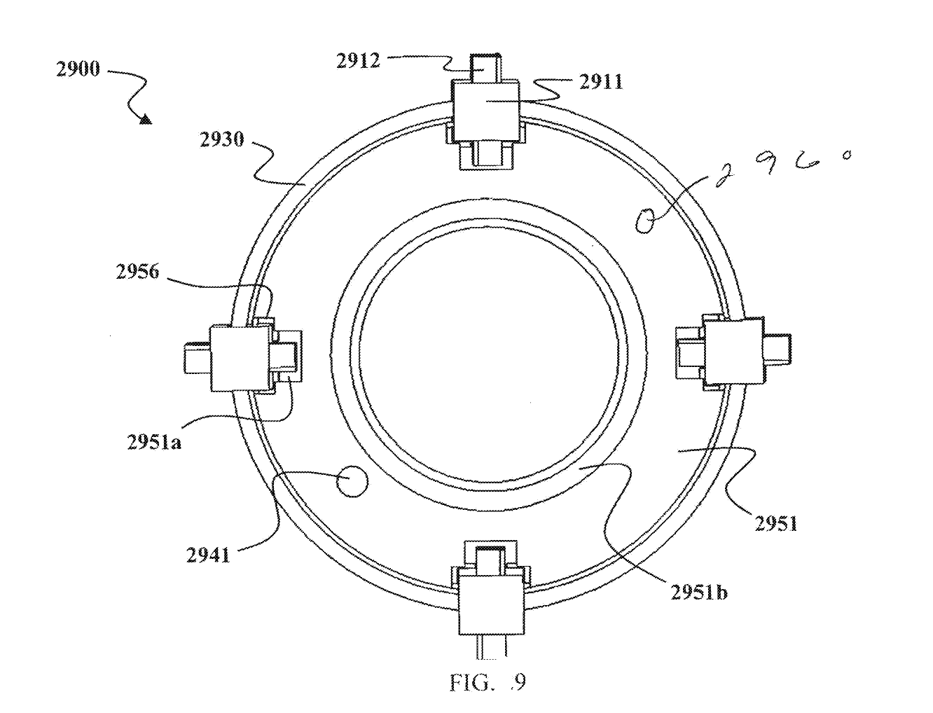

FIG. 9 shows a cross-sectional view of a portion of the drive unit, including the drive shaft, support for the functional drive, and cooperating belts. Drive unit 2900, in this embodiment, comprises supports 2951, which support the functional drive and in certain embodiments the endoscope. In this embodiment, proximal support 2951 is shown, which comprises groove 2951b for supporting the inner cylinder, which is concentric with the internal surface of the shaft of the functional drive and thus supports the functional drive. Drive shaft 2941 passes through proximal support 2951 and by way of gears engages the functional drive, which engages belts 2911 at tread 2912 (here, castellated projections). Belts 2911 rest on belt guides 2956 (which are supported by notches 2951a in supports 2951) and the belts are held in position (to maintain interaction with the functional drive) on the opposing side of the belts by cowling 2952. Membranous element 2930 (balloon) rests on and/or is secured to cowling 2952 and supports belts 2911, which circumscribe the balloon. As explained above, a hole 2960 for an air insufflations tube is provided.

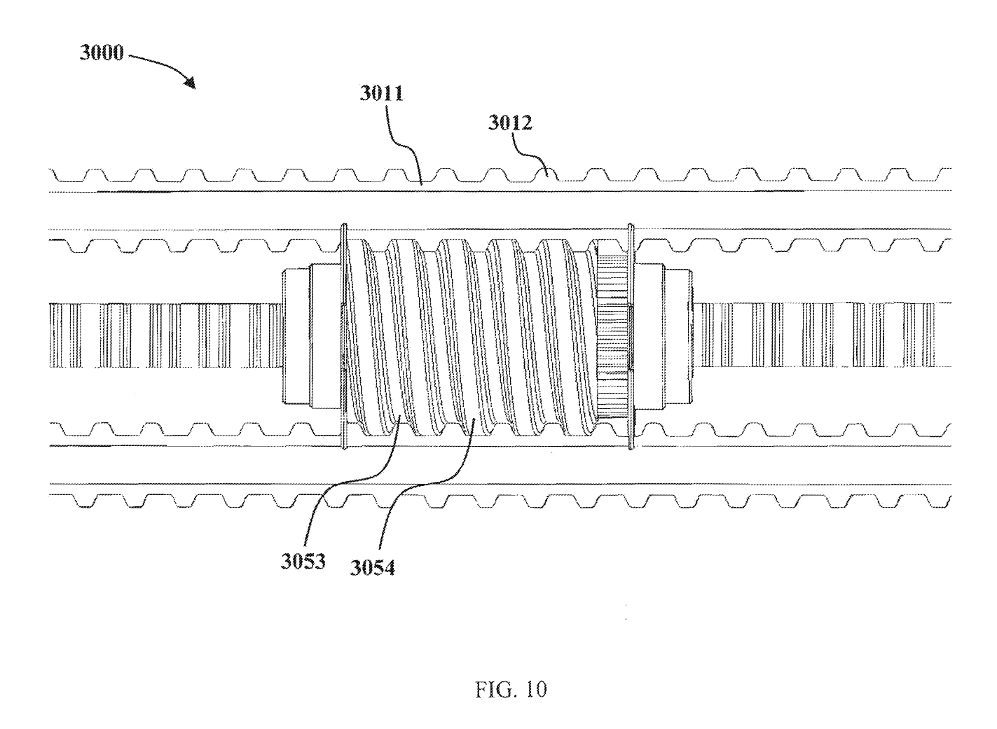

The functional drive and cooperating belts of the drive unit can have complementary surfaces that complement each other to any degree appropriate under the circumstances. For example, the functional drive can comprise any number of protruding structures for engagement with any number of protruding structures of the belts. Additionally, the cooperating surfaces of the functional drive and the belts need not be exactly complementary, only sufficiently complementary to provide means for engaging and rotating the belts. As shown in FIG. 10, the protruding castellated structures of functional drive and the cooperating belts of drive unit 3000 correspond on a one-to-one ratio and have surfaces that closely, though not necessarily exactly, complement one another. More specifically, belts 3011 comprise tread 3012, which engages with functional structure 3054 of functional drive 3053. As shown, these complementary castellated projections provide surfaces that are sufficiently complementary for engagement and rotation of the belts.

FIG. 11 shows a portion of drive unit 3100, including cowling 3152, drive shaft 3141, and supports 3151. In embodiments where the functional drive and corresponding belts have a high degree of complementing one another, supports 3151 can comprise square or U-shaped notches and need not comprise a shape that provides support for additional belt supports. For example, the embodiment shown in FIG. 11 can be contrasted with the embodiment of FIG. 5B, which shows T-shaped notches 2551a. As shown in FIG. 5B, T-shaped notches 2551a are one means for supporting belt supports 2556. Although belt supports 2556, as shown in FIG. 5, may be desirable in some embodiments, such belt supports 2556 are not required in any embodiment. Belt supports may be dispensed with, for example, as in FIG. 11. In the embodiment shown in FIG. 11, no additional belt supports are needed because the functional drive and cooperating belts complement one another to a high degree, which provides sufficient support for the belts. As explained above, a hole 3160 for an air insufflation tube is provided.

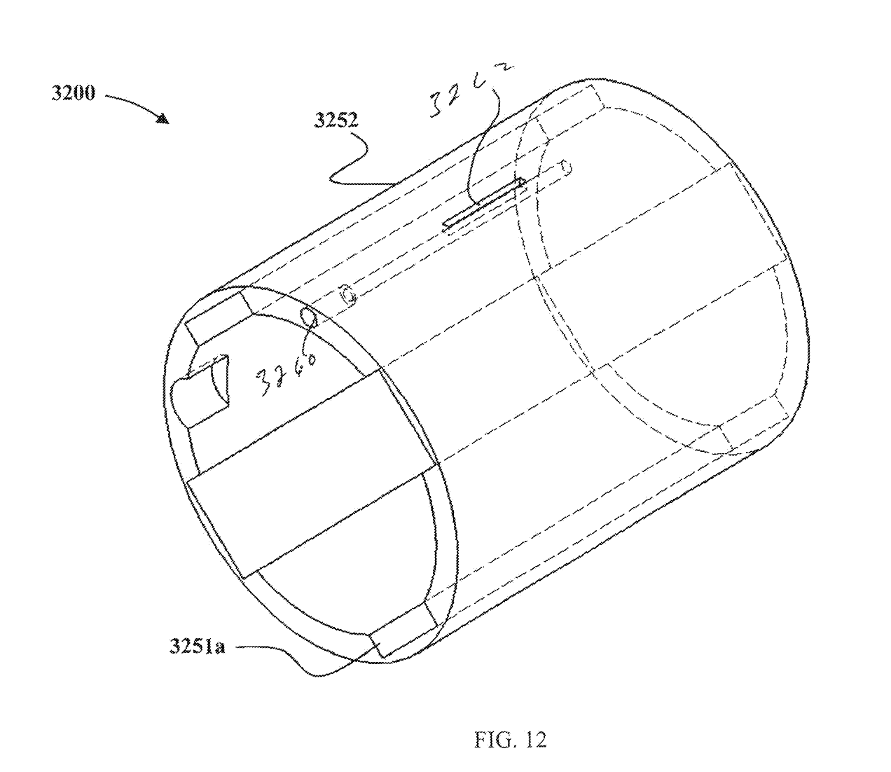

FIG. 12 shows a portion of drive unit 3200 according to one embodiment of the invention, in particular cowling 3252. Of particular interest about cowling 3252 are notches 3251a, which in this embodiment are U-shaped or square to accommodate belts and allow for the belts to rotate, while being supported by the cowling to provide sufficient resistance/guidance to the belts to keep them engaged with the functional drive. Such cowlings 3252 can be used in any embodiment of a drive unit according to the invention whether or not additional belt supports are also used. An air insufflation port 3260 connects to the cowling with an opening 3262 for air insufflation of the balloon.

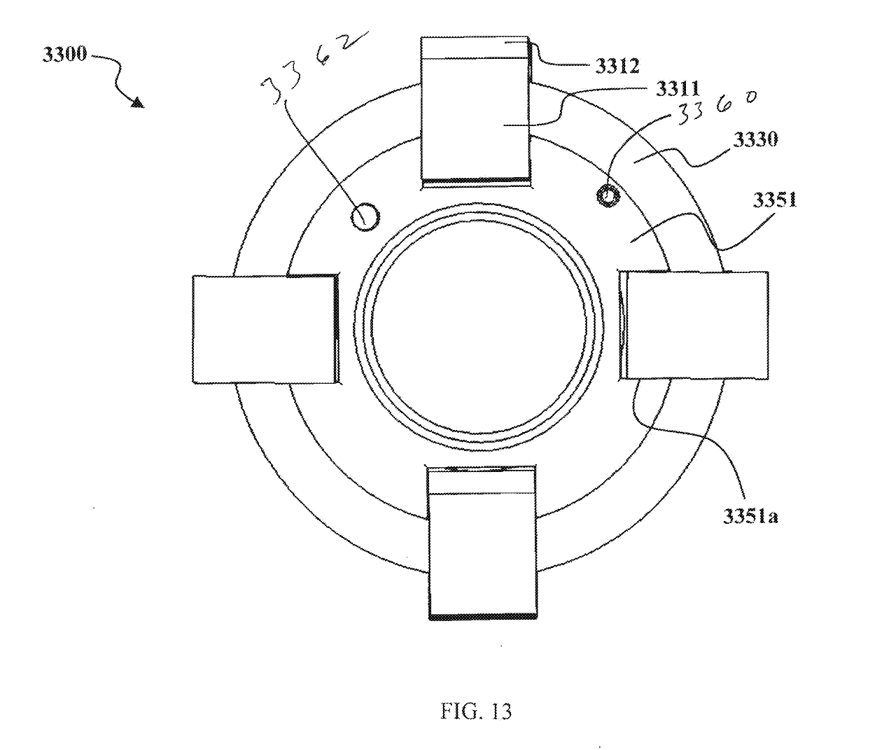

FIG. 13 shows a cross-sectional view of one embodiment of the drive unit 3300 according to the invention. As shown, supports 3351 comprise notches 3351a, which are U-shaped or square (as opposed to T-shaped). Such supports 3351 can be used in embodiments of the invention where additional belt supports are not needed. U-shaped notches 3351a provide for belts 3311 (for example, belts with tread 3312) to rotate freely around balloon 3330. It is desirable to use supports 3351 in embodiments where additional structure (as provided by, for example, T-shaped supports in combination with additional belt supports) is not needed to support belts 3311 along their length. Holes 3360, 3362 are provided for the drive shaft and the air insufflation tube, respectively.

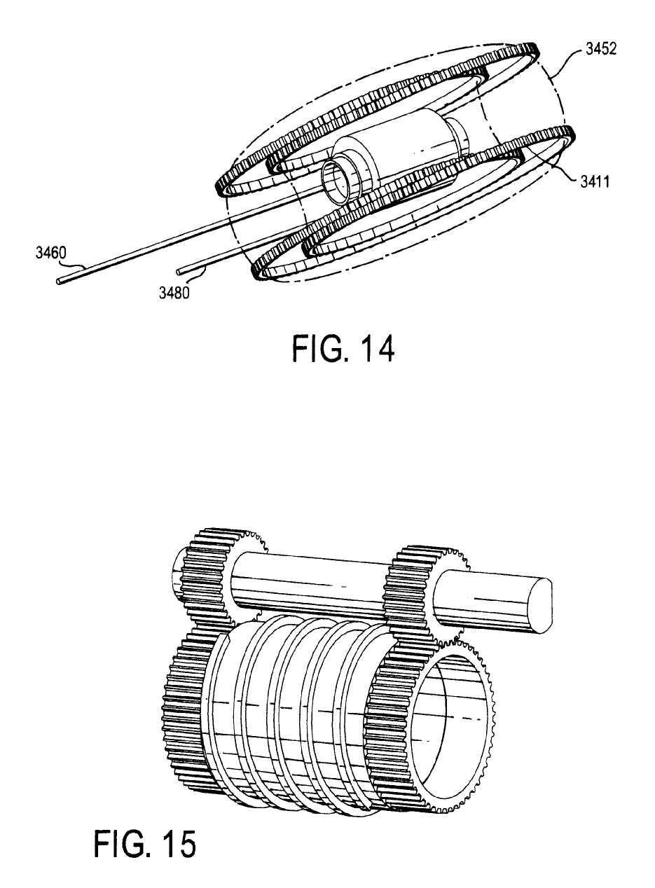

FIG. 14 shows one embodiment of a propulsion system according to the invention. This view is provided to show inflation of balloon 3452. As described above, inflation of the membranous element, or balloon, can be achieved in various ways, including with any fluid, such as air. As shown in this embodiment, fluid hose 3460 communicates with balloon 3452 for purposes of supplying fluid, such as air, to balloon 3452 for inflation. Inflation of balloon 3452 exerts pressure on belts 3411 for engaging a cavity wall in which the device is located. Fluid hose 3460 can be connected to balloon 3452 by any means, such as snap fitting, friction fit, or can be incorporated into balloon 3452 to provide the balloon and hose as a single component. A pressure transducer (not shown) can be used to inflate and deflate balloon 3452, as appropriate. For example, the pressure transducer can comprise a sensor for determining and evaluating an amount of pressure between balloon 3452 and the walls of a cavity, such as a colon, to automatically inflate or deflate the balloon to maintain an appropriate amount of pressure between the propulsion system and the cavity walls. Drive shaft 3480 is also shown.

Of course, one or more of the various features of the embodiments and aspects discussed above may be combined with one or more other features discussed above with respect to other embodiments and aspects to achieve particular configurations that are advantageous for a particular use. The combinations specifically described above simply depict exemplary embodiments, while the invention encompasses all combinations of elements and method steps to achieve all of the purposes disclosed herein or envisioned by those of skill in the art. It will thus be apparent to those skilled in the art that various modifications and variations can be made in the practice of the present invention and in construction of the device and medical instruments comprising the device without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

1. A propulsion device for an endoscope, the device comprising:
a rotatable drive shaft;
one or more flexible belts that releasably contact a cavity surface;
a membranous element, circumscribed by the belts, that exerts pressure on the belts in the direction of the cavity surface; and
a functional drive that rotates the belts in response to rotation of the drive shaft, the rotation of the belts causing longitudinal movement of the device along the cavity surface.

2. The device of claim 1, wherein the membranous element is configured to inflate and deflate to increase and decrease the pressure on the belts.

3. The device of claim 2, further comprising an insufflation tube in flow communication with the membranous element.

4. The device of claim 3, further comprising a pressure transducer that inflates the membranous element via the insufflation tube.

5. The device of claim 4, further comprising a sensor that determines an amount of pressure exerted on the cavity surface by the belts and/or the membranous element.

6. The device of claim 5, wherein the device automatically inflates and/or deflates the membranous element such that an appropriate amount of pressure is exerted on the cavity surface by the belts and/or the membranous element.

7. The device of claim 1, wherein the functional drive comprises a worm drive, a friction drive, a magnetic drive, or a direct gear drive.

8. The device of claim 1, wherein the belts include a tread and the functional drive includes a complementary structure that engages with the tread.

9. The device of claim 8, wherein the tread comprises castellated projections.

10. The device of claim 8, wherein the tread engages with the cavity surface and provides traction for the longitudinal movement of the device.

11. A method of propelling a device along a cavity surface, the method comprising:
providing a rotatable drive shaft;
providing at least one flexible belt that releasably contact the cavity surface;
providing a membranous element, circumscribed by the at least one belt;
providing a functional drive that rotates the at least one belt in response to rotation of the drive shaft;
inflating the membranous element such that the at least one belt exerts pressure on the at least one belt in the direction of the cavity surface; and
rotating the drive shaft such that the functional drive rotates the at least one belt and the rotation of the at least one belt propels the device along the cavity surface.

12. The method of claim 11, wherein inflating the membranous element comprises inflating the membranous element via an insufflation tube in flow communication with the membranous element.

13. The method of claim 12, wherein a pressure transducer inflates the membranous element via the insufflations tube.

14. The method of claim 12, wherein the pressure transducer is outside the cavity.

15. The method of claim 13, further comprising:
determining, by a sensor, an amount of pressure exerted on the cavity surface by the at least one belt and/or the membranous element.

16. The method of claim 15, further comprising:
automatically inflating and/or deflating the membranous element such that an appropriate amount of pressure is exerted on the cavity surface by the at least one belt and/or the membranous element.

17. The method of claim 11, wherein the functional drive comprises a worm drive, a friction drive, a magnetic drive, or a direct gear drive.

18. The method of claim 1, wherein at least one belt include a tread and the functional drive includes a complementary structure that engages with the tread.

19. The method of claim 18, wherein the tread comprises castellated projections.

20. The method of claim 18, wherein the tread engages with the cavity surface and provides traction for the longitudinal movement of the device.
